(19) United States
(12) Patent Application Publication
(10) Pub. No.: US 2025/0301210 A1
(43) Pub. Date: Sep. 25, 2025

(54) ENDOSCOPE SYSTEM AND OPERATION DEVICE

(71) Applicant: Evident Corporation, Kamiina-gun (JP)

(72) Inventor: Yukiyoshi YAGI, Kamiina-gun (JP)

(73) Assignee: Evident Corporation, Kamiina-gun (JP)

(21) Appl. No.: 19/231,061

(22) Filed: Jun. 6, 2025

Related U.S. Application Data
(63) Continuation of application No. PCT/JP2023/041081, filed on Nov. 15, 2023.

(30) Foreign Application Priority Data
Dec. 13, 2022 (JP) 2022-198676

(51) Int. Cl. H04N 23/50

(52) U.S. Cl. CPC H04N 23/555

(57) ABSTRACT

An endoscope system includes an endoscope device including a bendable insertion unit, an operation device, and a processor. A user interface of the operation device generates a first signal in accordance with a state of the user interface. The state changes when the user interface touches an object. A motion sensor of the operation device generates a second signal in accordance with physical movement of the operation device. The processor is configured to calculate a first control value used in first control based on the first signal. The processor is configured to calculate a second control value used in second control based on the second signal. At least one of the first control and the second control is executed to bend the insertion unit.

### BACKGROUND OF THE INVENTION

### Field of the Invention

The present invention relates to an endoscope system and an operation device.

Priority is claimed on Japanese Patent Application No. 2022-198676, filed Dec. 13, 2022 and is a continuation application based on PCT Patent Application No. PCT/JP2023/041081, filed on Nov. 15, 2023, and the content of both the Japanese patent application and the PCT patent application is incorporated herein by reference.

### DESCRIPTION OF RELATED ART

An Industrial endoscope device has been used for inspection of internal abnormalities, corrosion, and the like of boilers, pipes, aircraft engines, and the like. The endoscope device includes an insertion unit used for acquiring an image. A user inserts the insertion unit into a subject and acquires an image of an inspection portion in the subject. The user observes the image and inspects the inspection portion. The insertion unit includes a bending portion that bends the insertion unit. The user can bend the insertion unit by performing a bending operation.

A technique disclosed in Japanese Unexamined Patent Application, First Publication No. 2019-000352 provides a function of determining an instruction on a predetermined motion based on a rotation amount or a movement amount of a housing (casing) of a remote controller or the like. For example, a smartphone is used as the remote controller. In an example disclosed in Japanese Unexamined Patent Application, First Publication No. 2019-000352, a user can simultaneously input a bending instruction in a normal mode and a bending instruction in a fine mode by rotating or moving the housing. When the bending instruction in the normal mode is stopped, a distal end of an insertion unit returns to an initial position. When the bending instruction in the fine mode is stopped, the distal end of the insertion unit does not return to the initial position and the insertion unit keeps being bent.

### SUMMARY OF THE INVENTION

According to a first aspect of the present invention, an endoscope system includes an endoscope device including a bendable insertion unit, an operation device, and a processor. The operation device includes a housing, a user interface, and a motion sensor. The user interface is disposed on the housing and generates a first signal in accordance with a state of the user interface. The state changes when the user interface touches an object. The motion sensor is configured to generate a second signal in accordance with physical movement of the operation device. The processor is configured to calculate a first control value used in first control of the endoscope device based on the first signal. The processor is configured to calculate a second control value used in second control of the endoscope device based on the second signal. At least one of the first control and the second control is executed to bend the insertion unit.

According to a second aspect of the present invention, in the first aspect, the first control and the second control may be executed to bend the insertion unit.

According to a third aspect of the present invention, in the second aspect, a maximum value of a bending amount of the insertion unit in the second control may be different from a maximum value of the bending amount of the insertion unit in the first control.

According to a fourth aspect of the present invention, in the second aspect, the processor may be configured to calculate the first control value in a first period during which the first signal is generated without the second signal being generated. The processor may be configured to calculate the first control value without calculating the second control value in a second period during which the first signal and the second signal are generated after the first period.

According to a fifth aspect of the present invention, in the second aspect, the processor may be configured to calculate the second control value in a first period during which the second signal is generated without the first signal being generated. The processor may be configured to calculate the second control value without calculating the first control value in a second period during which the first signal and the second signal are generated after the first period.

According to a sixth aspect of the present invention, in the second aspect, a range of a bending amount of the insertion unit in the second control may be different from a range of the bending amount of the insertion unit in the first control.

According to a seventh aspect of the present invention, in the first aspect, the physical movement may include rotation of the operation device.

According to an eighth aspect of the present invention, in the seventh aspect, the motion sensor may be configured to generate a third signal in accordance with the rotation of the operation device. The processor may be configured to allocate one of control of a first function and control of a second function to the first control based on the third signal. The processor may be configured to allocate the other of the control of the first function and the control of the second function to the second control based on the third signal.

According to a ninth aspect of the present invention, in the seventh aspect, the operation device may have an elongated shape. The motion sensor may be configured to generate the second signal in accordance with the rotation of the operation device around an axis perpendicular to a longitudinal direction of the operation device. The second control may be executed to bend the insertion unit.

According to a tenth aspect of the present invention, in the seventh aspect, the operation device may include a sensor that outputs a signal in accordance with whether a user is holding the operation device with a left hand or is holding the operation device with a right hand. The processor may be configured to determine whether the user is holding the operation device with the left hand or is holding the operation device with the right hand based on the signal output from the sensor. The processor may be configured to calculate the second control value by using first information indicating a relationship between an amount of the rotation and the second control value when it is determined that the user is holding the operation device with the left hand. The processor may be configured to calculate the second control value by using second information indicating a relationship between the amount of the rotation and the second control value when it is determined that the user is holding the operation device with the right hand. The second information may be different from the first information.

According to an eleventh aspect of the present invention, in the first aspect, the endoscope system may further include an insertion device that inserts a distal end of the insertion unit into a subject and pulls the distal end out of the subject. One of the first control and the second control may be executed to bend the insertion unit. The other of the first control and the second control may be executed to control insertion and pullout of the distal end.

According to a twelfth aspect of the present invention, in the eleventh aspect, the operation device may have an elongated shape. The motion sensor may be configured to generate a third signal in accordance with rotation of the operation device around an axis which is parallel to a longitudinal direction of the operation device. The insertion device may rotate the insertion unit around a center axis of the insertion unit. The processor may be configured to calculate, based on the third signal, a third control value used to control rotation of the insertion unit.

According to a thirteenth aspect of the present invention, in the first aspect, the operation device may include a switch disposed on the housing. A state of the switch may be switched between a first state and a second state. The second signal may be valid when the state of the switch is the first state. The second signal may be invalid when the state of the switch is the second state. The processor may be configured to calculate the second control value only when the second signal is valid.

According to a fourteenth aspect of the present invention, in the first aspect, the user interface may include a movable member disposed on the housing.

According to a fifteenth aspect of the present invention, in the first aspect, the operation device may include the processor.

According to a sixteenth aspect of the present invention, in the first aspect, the endoscope device may include the processor.

According to a seventeenth aspect of the present invention, in the first aspect, the endoscope device may include an image sensor configured to generate an image based on an optical image acquired by the insertion unit. One of the first control and the second control is executed to control image processing of changing a state of the image.

According to an eighteenth aspect of the present invention, in the first aspect, the processor may be configured to select one of three or more control values corresponding to three or more bending amounts of the insertion unit as the first control value based on the first signal.

According to a nineteenth aspect of the present invention, in the first aspect, the user interface may be disposed on a surface of the housing. The processor may be configured to convert the second signal generated in accordance with the movement of the operation device in a first state into the second signal generated in accordance with the movement of the operation device in a second state. A straight line perpendicular to the surface is not parallel to a gravitational direction in the first state. The straight line is parallel to the gravitational direction in the second state.

According to a twentieth aspect of the present invention, in the first aspect, the user interface may be disposed on a surface of the housing. The processor may be configured to convert the second signal generated in accordance with the movement of the operation device in a first state into the second signal generated in accordance with the movement of the operation device in a second state. A straight line parallel to the surface is not perpendicular to a gravitational direction in the first state. The straight line is perpendicular to the gravitational direction in the second state.

According to a twenty-first aspect of the present invention, an operation device includes a housing, a user interface, and a motion sensor. The user interface is disposed on the housing and generates a first signal in accordance with a state of the user interface. The state changes when the user interface touches an object. The motion sensor generates a second signal in accordance with physical movement of the operation device. The first signal is used to calculate a first control value used in first control of an endoscope device including a bendable insertion unit. The second signal is used to calculate a second control value used in second control of the endoscope device. At least one of the first control and the second control is executed to bend the insertion unit.

### BRIEF DESCRIPTION OF THE DRAWINGS

FIG. 1 is a block diagram showing a configuration of an endoscope system according to a first embodiment of the present invention.

FIG. 2 is a front view and a side view of an operation device included in the endoscope system according to the first embodiment of the present invention.

FIG. 3 is a side view of the operation device included in the endoscope system according to the first embodiment of the present invention.

FIG. 4 is a front view and a side view of the operation device included in the endoscope system according to the first embodiment of the present invention.

FIG. 5 is a block diagram showing a configuration of the endoscope system related to bending control in the first embodiment of the present invention.

FIG. 6 is a diagram showing an example of a relationship between operations performed by a user and details of the bending control according to the first embodiment of the present invention.

FIG. 7 is a diagram showing an example of a relationship between operations performed by a user and details of the bending control according to the first embodiment of the present invention.

FIG. 8 is a diagram showing an example of a relationship between operations performed by a user and details of the bending control according to the first embodiment of the present invention.

FIG. 9 is a diagram showing an example of a relationship between operations performed by a user and details of the bending control according to the first embodiment of the present invention.

FIG. 10 is a diagram showing an example of a relationship between operations performed by a user and details of the bending control according to the first embodiment of the present invention.

FIG. 11 is a diagram showing a range in which a bending unit included in the endoscope system according to the first embodiment of the present invention can be bent.

FIG. 12A is a diagram showing an example of a state of the operation device in the first embodiment of the present invention.

FIG. 12B is a diagram showing an example of a bending control value calculated in accordance with an operation of a joystick in the first embodiment of the present invention.

FIG. 13A is a diagram showing an example of a state of the operation device in the first embodiment of the present invention.

FIG. 13B is a diagram showing an example of a bending control value calculated in accordance with an operation of the joystick in the first embodiment of the present invention.

FIG. 14A is a diagram showing an example of a bending control value calculated in accordance with an operation of the joystick in the first embodiment of the present invention.

FIG. 14B is a diagram showing an example of a bending control value calculated in accordance with an operation of the joystick in the first embodiment of the present invention.

FIG. 15A is a diagram showing an example of a state of the operation device in the first embodiment of the present invention.

FIG. 15B is a diagram showing an example of a bending control value calculated in accordance with a tilted state of the operation device according to the first embodiment of the present invention.

FIG. 16A is a diagram showing an example of a state of the operation device in the first embodiment of the present invention.

FIG. 16B is a diagram showing an example of a bending control value calculated in accordance with a tilted state of the operation device in the first embodiment of the present invention.

FIG. 17A is a diagram showing an example of a state of the operation device in the first embodiment of the present invention.

FIG. 17B is a diagram showing an example of a bending control value calculated in accordance with a tilted state of the operation device in the first embodiment of the present invention.

FIG. 18A is a diagram showing an example of a bending control value calculated in accordance with a tilted state of the operation device in a minute movement mode in the first embodiment of the present invention.

FIG. 18B is a diagram showing an example of a bending control value calculated in accordance with a tilted state of the operation device in the minute movement mode in the first embodiment of the present invention.

FIG. 19 is a diagram showing an example of a bending control value calculated in accordance with a tilted state of the operation device in the first embodiment of the present invention.

FIG. 20A is a diagram showing an example of a state of the operation device in the first embodiment of the present invention.

FIG. 20B is a diagram showing an example of a bending control value calculated in accordance with an operation of the joystick and a tilted state of the operation device in the first embodiment of the present invention.

FIG. 21A is a diagram showing an example of a state of the operation device in the first embodiment of the present invention.

FIG. 21B is a diagram showing an example of a bending control value calculated in accordance with an operation of the joystick and a tilted state of the operation device in the first embodiment of the present invention.

FIG. 22A is a diagram showing an example of a state of the operation device in the first embodiment of the present invention.

FIG. 22B is a diagram showing an example of a bending control value calculated in accordance with an operation of the joystick and a tilted state of the operation device in the first embodiment of the present invention.

FIG. 23 is a diagram showing an example of a bending control value calculated in accordance with an operation of the joystick and a tilted state of the operation device in the first embodiment of the present invention.

FIG. 24A is a diagram showing an example of a state of the operation device in the first embodiment of the present invention.

FIG. 24B is a diagram showing an example of a bending control value calculated in accordance with an operation of the joystick and a tilted state of the operation device in the first embodiment of the present invention.

FIG. 25 is a diagram showing an example of a bending control value calculated in accordance with an operation of the joystick and a tilted state of the operation device in the first embodiment of the present invention.

FIG. 26A is a graph showing an example of a relationship between an angle of the joystick and a bending control value in the first embodiment of the present invention.

FIG. 26B is a graph showing an example of a relationship between an angle of the joystick and a bending control value in the first embodiment of the present invention.

FIG. 27 is a front view of an operation device included in an endoscope system according to a second embodiment of the present invention.

FIG. 28 is a block diagram showing a configuration of an endoscope system according to a third embodiment of the present invention.

FIG. 29A is a diagram showing an example of a relationship between operations performed by a user and details of control in the third embodiment of the present invention.

FIG. 29B is a diagram showing an example of a relationship between operations performed by a user and details of control in the third embodiment of the present invention.

FIG. 30 is a front view and a side view of an operation device included in the endoscope system according to the third embodiment of the present invention.

FIG. 31 is a block diagram showing a configuration of an endoscope system according to a fourth embodiment of the present invention.

FIG. 32 is a front view of an operation device included in the endoscope system according to the fourth embodiment of the present invention.

FIG. 33A is a graph showing an example of first bending information in the fourth embodiment of the present invention.

FIG. 33B is a graph showing an example of second bending information in the fourth embodiment of the present invention.

FIG. 34 is a diagram showing an example of a relationship between operations performed by a user and details of control in a fifth embodiment of the present invention.

FIG. 35 is a diagram showing an example of a relationship between operations performed by a user and details of control in the fifth embodiment of the present invention.

FIG. 36 is a diagram showing an example of a relationship between operations performed by a user and details of control in the fifth embodiment of the present invention.

FIG. 37 is a block diagram showing a configuration of an endoscope system according to a sixth embodiment of the present invention.

FIG. 38 is a diagram showing an example of a relationship between operations performed by a user and details of control in the sixth embodiment of the present invention.

FIG. 39 is a diagram showing an example of a relationship between operations performed by a user and details of control in a reference embodiment of the present invention.

FIG. 40 is a graph showing an example of a first image-processing control value and a second image-processing control value in the reference embodiment of the present invention.

FIG. 41 is a diagram showing an example of a relationship between operations performed by a user and details of control in the reference embodiment of the present invention.

FIG. 42 is a graph showing an example of a first image-processing control value, a second image-processing control value, and a third image-processing control value in the reference embodiment of the present invention.

FIG. 43A is a diagram showing an example of a state of the operation device in the first embodiment of the present invention.

FIG. 43B is a diagram showing an example of a state of the operation device in the first embodiment of the present invention.

FIG. 44 is a diagram showing an example of a state of an insertion unit included in the endoscope system according to the first embodiment of the present invention.

FIG. 45A is a diagram showing an example of a state of the operation device in the first embodiment of the present invention.

FIG. 45B is a diagram showing an example of a state of the operation device in the first embodiment of the present invention.

FIG. 46A is a diagram showing an example of a state of the operation device in the first embodiment of the present invention.

FIG. 46B is a diagram showing an example of a state of the operation device in the first embodiment of the present invention.

FIG. 47 is a diagram showing an example of a state of the insertion unit included in the endoscope system according to the first embodiment of the present invention.

FIG. 48A is a diagram showing an example of a state of an operation device in a seventh embodiment of the present invention.

FIG. 48B is a diagram showing an example of a state of the operation device in the seventh embodiment of the present invention.

FIG. 49A is a diagram showing an example of a bending control value calculated in accordance with a tilted state of the operation device in a coarse movement mode in the seventh embodiment of the present invention.

FIG. 49B is a diagram showing an example of a bending control value calculated in accordance with a tilted state of the operation device in a minute movement mode in the seventh embodiment of the present invention.

FIG. 50A is a diagram showing an example of a state of an operation device in a seventh embodiment of the present invention.

FIG. 50B is a diagram showing an example of a state of the operation device in the seventh embodiment of the present invention.

### DETAILED DESCRIPTION OF THE INVENTION

Hereinafter, embodiments of the present invention will be described with reference to the drawings.

### First Embodiment

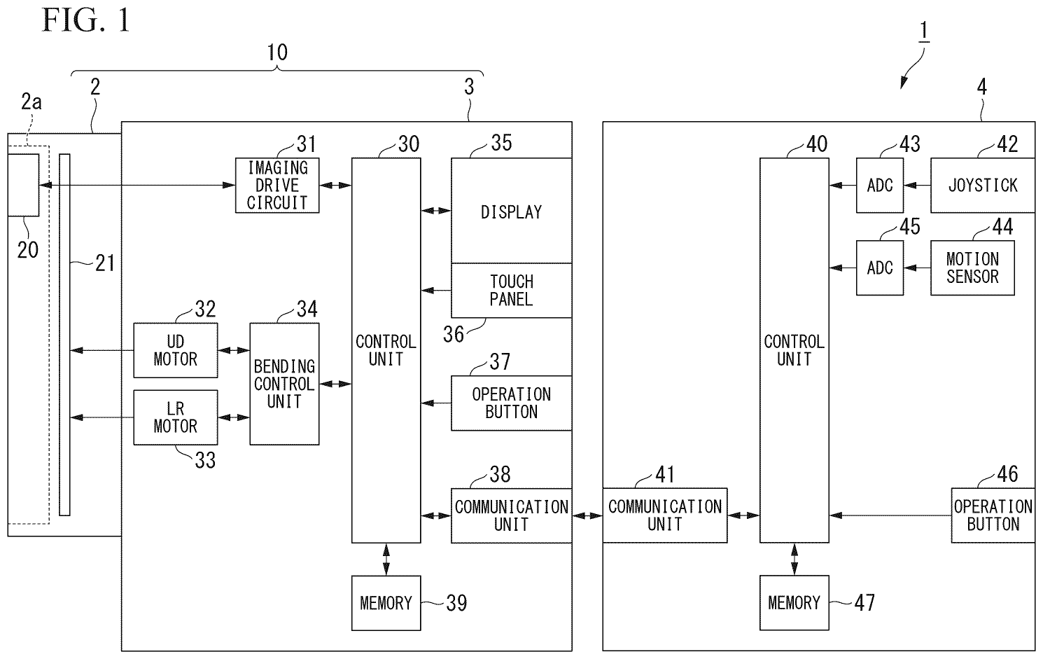

FIG. 1 shows a configuration of an endoscope system 1 according to a first embodiment of the present invention. The endoscope system 1 shown in FIG. 1 includes an insertion unit 2, a main body unit 3, and an operation device 4. The insertion unit 2 and the main body unit 3 constitute an endoscope device 10.

The insertion unit 2 is to be inserted into the inside of a subject. The insertion unit 2 has an elongated tubular shape and is bendable. A user performs an insertion operation and inserts the insertion unit 2 into the subject. The insertion unit 2 acquires an optical image of the inside of the subject. The insertion unit 2 includes an imaging unit 20 and a bending portion 21.

The imaging unit 20 is disposed in a distal end portion 2a including a distal end of the insertion unit 2. The imaging unit 20 is an image sensor such as a charge-coupled device (CCD) sensor or a complementary metal-oxide-semiconductor (CMOS) sensor. The imaging unit 20 generates an image based on the optical image acquired by the insertion unit 2. The image generated by the imaging unit 20 is output to the main body unit 3. The bending portion 21 bends the insertion unit 2 upward, downward, leftward, or rightward.

The main body unit 3 includes a control unit 30, an imaging drive circuit 31, a UD motor 32, an LR motor 33, a bending control unit 34, a display 35, a touch panel 36, an operation button 37, a communication unit 38, and a memory 39. Part of the main body unit 3 may be constituted by a tablet terminal or the like. For example, the tablet terminal may include part of the main body unit 3 (the display 35 and the like).

The control unit 30 controls each unit of the main body unit 3. The control unit may be constituted by at least one of a processor and a logic circuit. For example, the processor is at least one of a central processing unit (CPU), a digital signal processor (DSP), and a graphics-processing unit (GPU). For example, the logic circuit is at least one of an application-specific integrated circuit (ASIC) and a field-programmable gate array (FPGA). The control unit 30 may include one or more processors. The control unit 30 may include one or more logic circuits.

A computer of the endoscope system 1 may read a program and execute the read program. The program includes commands defining the operations of the control unit 30. In other words, the functions of the control unit 30 may be realized by software.

The program described above, for example, may be provided by using a “computer-readable recording medium” such as a flash memory. The program may be transmitted from the computer storing the program to the endoscope system 1 through a transmission medium or transmission waves in a transmission medium. The “transmission medium” transmitting the program is a medium having a function of transmitting information. The medium having the function of transmitting information includes a network (communication network) such as the Internet and a communication circuit line (communication line) such as a telephone line. The program described above may realize some of the functions described above. In addition, the program described above may be a differential file (differential program). The functions described above may be realized by a combination of a program that has already been recorded in a computer and a differential program.

The imaging drive circuit 31 controls the imaging unit 20 and outputs the image output from the imaging unit 20 to the control unit 30.

The UD motor 32 is connected to a UD bending wire used for bending the bending portion 21 upward or downward. The UD motor 32 bends the bending portion 21 upward or downward by pulling the UD bending wire. The LR motor 33 is connected to an LR bending wire used for bending the bending portion 21 leftward or rightward. The LR motor 33 bends the bending portion 21 leftward or rightward by pulling the LR bending wire. The bending control unit 34 controls the UD motor 32 and the LR motor 33.

The display 35 is a monitor such as a liquid crystal display (LCD). The display displays an image generated by the imaging unit 20. The touch panel 36 is disposed on the screen of the display 35. A user can input, for example, an instruction to change the setting of the endoscope system 1 to the endoscope system 1 by operating the touch panel 36. The operation button 37 receives various instructions from the user. The user can input various instructions to the endoscope system 1 by pressing the operation button 37. The communication unit 38 executes wired communication or wireless communication with a communication unit 41 included in the operation device 4.

The memory 39 is a nonvolatile recording medium. For example, the memory 39 is at least one of a static random-access memory (SRAM), an erasable programmable read-only memory (EPROM), an electrically erasable programmable read only memory (EEPROM), and a flash memory. The memory 39 may be attachable to and detachable from to the main body unit 3. For example, the memory 39 stores an image generated by the imaging unit 20 and various kinds of information processed by the control unit 30.

The operation device 4 is separate from the main body unit 3. The operation device 4 includes a control unit 40, a communication unit 41, a joystick 42, an analog-to-digital converter (ADC) 43, a motion sensor 44, an ADC 45, an operation button 46, and a memory 47. The operation device 4 functions as a remote controller. The operation device 4 may be an information terminal such as a smartphone or a tablet terminal.

The control unit 40 controls each unit of the operation device 4. The control unit 40 may be constituted by at least one of a processor and a logic circuit. The control unit 40 may include one or more processors. The control unit 40 may include one or more logic circuits. The computer of the endoscope system 1 may read a program and execute the read program. The program includes commands defining the operations of the control unit 40. In other words, the functions of the control unit 40 may be realized by software. The program realizing the functions of the control unit 40 may be realized similarly to the program realizing the functions of the control unit 30.

The communication unit 41 executes wired communication or wireless communication with the communication unit 38 of the main body unit 3.

The joystick 42 is a rod-like movable member and functions as a physical user interface. A user touches the joystick 42 with the finger or the like and applies a force to the joystick 42. By doing this, the user can tilt the joystick 42 upward, downward, leftward, or rightward. The joystick 42 outputs an analog voltage in accordance with a direction in which the joystick 42 is tilted and an angle by which the joystick 42 is tilted. The ADC 43 converts the analog voltage output from the joystick 42 into a digital value. The joystick 42 may include the ADC 43 and may output a digital value.

The posture of the operation device 4 changes in accordance with physical movement of the operation device 4. The movement is rotation or parallel translation. A user holds and moves the operation device 4. For example, the motion sensor 44 is a combination of an acceleration sensor and a gyro sensor. The motion sensor 44 determines movement of the operation device 4 and outputs an analog voltage in accordance with the movement. The ADC 45 converts the analog voltage output from the motion sensor 44 into a digital value. The motion sensor 44 may include the ADC and output a digital value.

The digital value output from the ADC 43 is used for first control of the endoscope device 10. The digital value output from the ADC 45 is used for second control of the endoscope device 10. At least one of the first control and the second control is bending control of bending the insertion unit 2. In the first embodiment, an example in which the first control and the second control are bending control will be described.

Two or more modes related to bending control are prepared. For example, a normal mode and a fine mode are used. Alternatively, a coarse movement mode and a minute movement mode described later are used.

In the first embodiment, a user can bend the insertion unit 2 by tilting the joystick 42 or moving the operation device 4. The user may tilt the joystick 42 and may move the operation device 4 at the same time.

The operation button 46 receives various instructions from a user. The user can input various instructions to the endoscope system 1 by pressing the operation button 46.

The memory 47 is a nonvolatile recording medium. For example, the memory 47 is at least one of an SRAM, an EPROM, an EEPROM, and a flash memory. The memory 47 stores various kinds of information processed by the control unit 40.

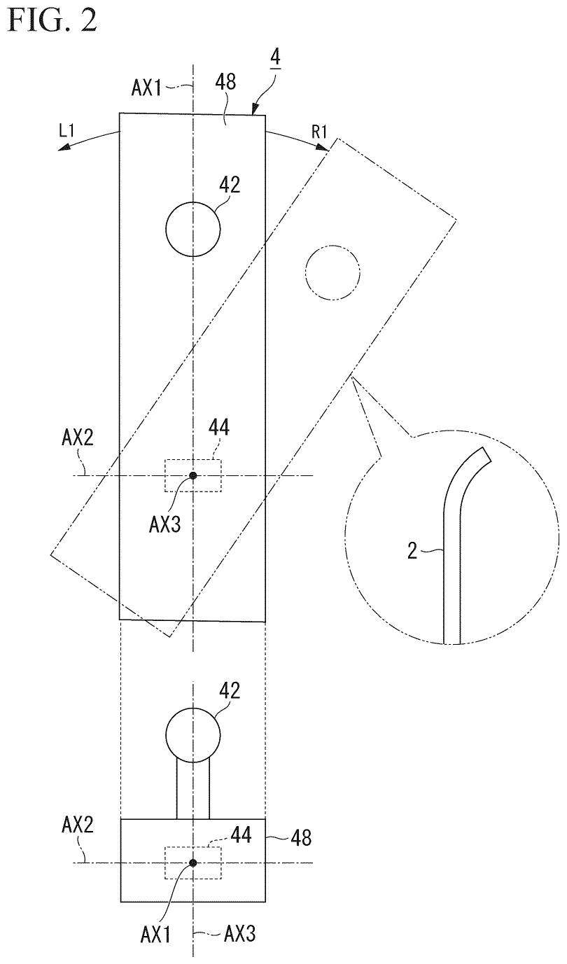

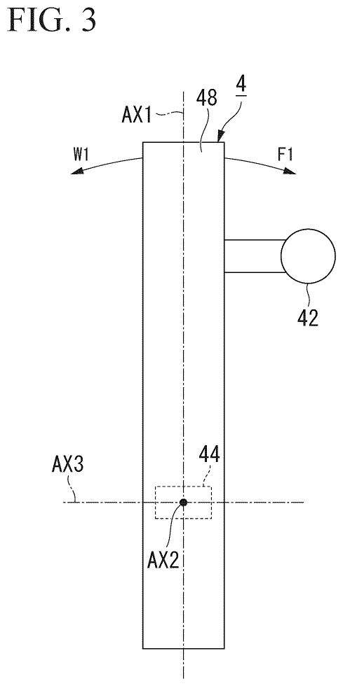

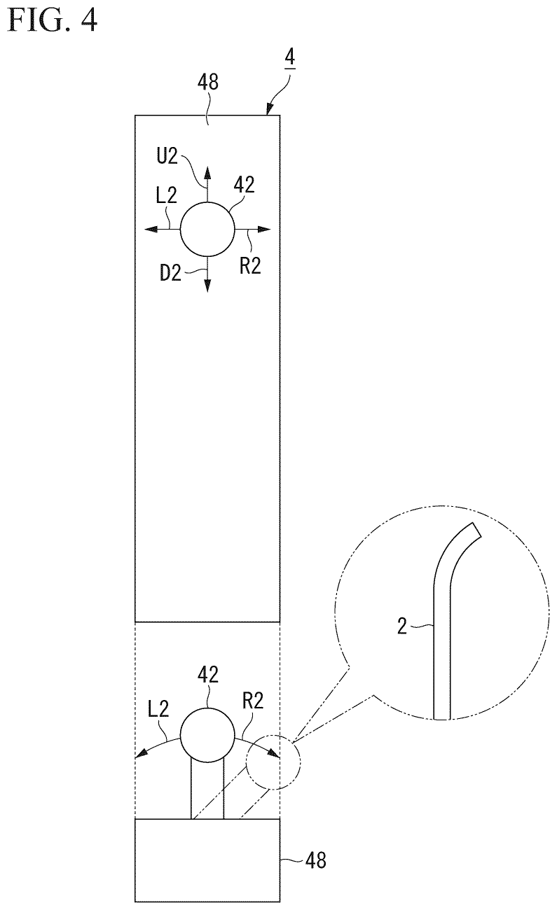

FIG. 2, FIG. 3, and FIG. 4 show an external appearance of the operation device 4. FIG. 2 is a front view and a side view of the operation device 4. The operation device 4 shown in FIG. 2 has an elongated shape. The shape of the operation device 4 is not limited to the example shown in FIG. 2.

The operation device 4 includes a housing 48. The joystick 42 is disposed on the housing 48. When a user is not operating the joystick 42, the joystick 42 is almost perpendicular to the surface of the operation device 4. The control unit 40, the communication unit 41, the ADC 43, the motion sensor 44, the ADC 45, and the memory 47 shown in FIG. 1 are disposed in the housing 48. The operation button 46 shown in FIG. 1 is not shown in FIG. 2.

The motion sensor 44 is fixed to the housing 48. The motion sensor 44 determines a rotation direction and a rotation amount of the housing 48 around each of an axis AX1, an axis AX2, and an axis AX3. The axis AX1, the axis AX2, and the axis AX3 are orthogonal to each other. The axis AX1 passes through a reference position of the motion sensor 44 and extends in a longitudinal direction of the housing 48. For example, the reference position is the center of the motion sensor 44. The axis AX2 and the axis AX3 pass through the reference position and are perpendicular to the axis AX1. The axis AX2 is parallel to the surface of the housing 48 on which the joystick 42 is provided. The axis AX3 is perpendicular to the surface of the housing 48 on which the joystick 42 is disposed.

For example, a user tilts the operation device 4 in a left direction L1 or a right direction R1. At this time, the motion sensor 44 determines a rotation direction and a rotation amount of the housing 48 around the axis AX3. When the user tilts the operation device 4 in the left direction L1, the insertion unit 2 is bent leftward. When a user tilts the operation device 4 in the right direction R1, the insertion unit 2 is bent rightward.

FIG. 3 is a side view of the operation device 4. For example, a user tilts the operation device 4 in a front direction F1 or a rear direction W1. At this time, the motion sensor 44 determines a rotation direction and a rotation amount of the housing 48 around the axis AX2. When the user tilts the operation device 4 in the front direction F1, the insertion unit 2 is bent upward. When the user tilts the operation device 4 in the rear direction W1, the insertion unit 2 is bent downward.

As described above, a user can intuitively bend the insertion unit 2 by tilting the operation device 4. Hereinafter, an operation in which the user tilts the operation device 4 is referred to as a tilting operation. The endoscope system 1 executes the second control in accordance with the tilting operation. As described above, the second control in the first embodiment is the bending control.

The motion sensor 44 may determine a movement direction and a movement amount related to translational movement of the housing 48 parallel to each of the axis AX1, the axis AX2, and the axis AX3. The endoscope system 1 may execute the first control or the second control based on the movement direction and the movement amount.

A direction (bending direction) in which the insertion unit 2 is bent corresponds to a direction in an image generated by the imaging unit 20. For example, when a user tilts the operation device 4 in the left direction L1, the insertion unit 2 is bent toward an object seen on the left side in the image.

FIG. 4 is a front view and a side view of the operation device 4. A user tilts the joystick 42 in an upward direction U2, a downward direction D2, a left direction L2, or a right direction R2. At this time, the joystick 42 outputs an analog voltage in accordance with a direction in which the joystick 42 is tilted and an angle by which the joystick 42 is tilted.

When a user tilts the joystick 42 in the upward direction U2, the insertion unit 2 is bent upward. When a user tilts the joystick 42 in the downward direction D2, the insertion unit 2 is bent downward. When a user tilts the joystick 42 in the left direction L2, the insertion unit 2 is bent leftward. When a user tilts the joystick 42 in the right direction R2, the insertion unit 2 is bent rightward.

A user can adjust the angle of the joystick 42 relatively easily. Therefore, the user can finely adjust a bending amount of the insertion unit 2. Hereinafter, an operation in which the user tilts the joystick 42 is referred to as a joystick operation. The endoscope system 1 executes the first control in accordance with the joystick operation. As described above, the first control in the first embodiment is the bending control.

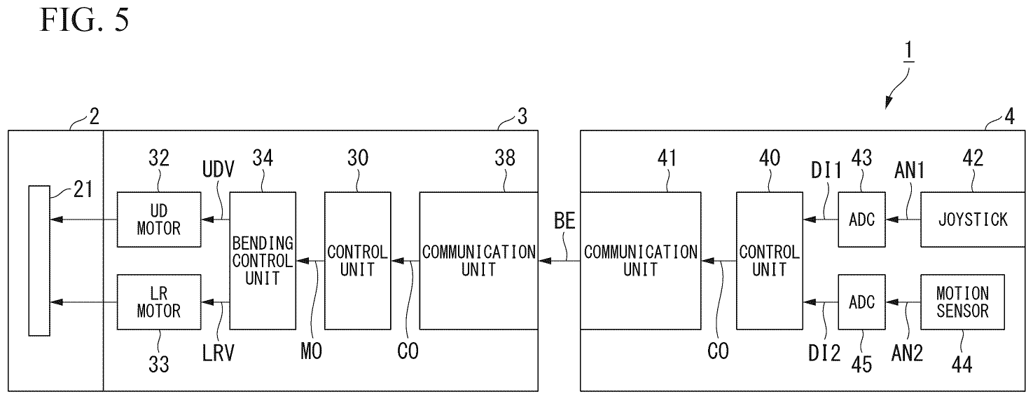

FIG. 5 shows a configuration of the endoscope system 1 related to the bending control. An unnecessary configuration for the bending control is not shown in FIG. 5.

The joystick 42 outputs an analog voltage AN1 (first signal) n accordance with a direction in which the joystick 42 is tilted and an angle by which the joystick 42 is tilted. For example, a range of the analog voltage AN1 is 0 [V] to V1 [V]. The voltage value V1 is larger than 0 [V]. The ADC 43 converts the analog voltage AN1 into a digital value DI1 and outputs the digital value DI1 to the control unit 40. For example, a range of the digital value DI1 is 000 h to 3 FFh in hexadecimal. The range of the analog voltage AN1 and the range of the digital value DI1 are not limited to the above-described examples.

The motion sensor 44 outputs an analog voltage AN2 (second signal) in accordance with a rotation direction and a rotation amount of the housing 48. For example, a range of the analog voltage AN2 is 0 [V] to V1 [V]. The ADC 45 converts the analog voltage AN2 into a digital value DI2 and outputs the digital value DI2 to the control unit 40. For example, a range of the digital value DI2 is 000 h to 3 FFh in hexadecimal. The range of the analog voltage AN2 and the range of the digital value DI2 are not limited to the above-described examples.

The control unit 40 calculates a bending control value CO based on the digital value DI1 and the digital value DI2. The bending control value CO indicates a bending direction and a bending amount of the insertion unit 2. The control unit 40 outputs the bending control value CO to the communication unit 41. The communication unit 41 transmits the bending control value CO to the communication unit 38. The communication unit 38 outputs the bending control value CO to the control unit 30.

The control unit 30 calculates a motor control value MO for driving at least one of the UD motor 32 and the LR motor 33 based on the bending control value CO. The motor control value MO indicates a driving amount of each motor. The control unit 30 may calculate a motor control value MO used for driving only the UD motor 32 or the LR motor 33. Alternatively, the control unit 30 may calculate a motor control value MO used for driving the UD motor 32 and the LR motor 33. The control unit 30 outputs the motor control value MO to the bending control unit 34.

The bending control unit 34 generates at least one of a control signal UDV and a control signal LRV based on the motor control value MO. The bending control unit 34 may generate only the control signal UDV or the control signal LRV. Alternatively, the bending control unit 34 may generate the control signal UDV and the control signal LRV. The control signal UDV includes a voltage and a current used for driving the UD motor 32. The control signal LRV includes a voltage and a current used for driving the LR motor 33. The bending control unit 34 outputs the control signal UDV to the UD motor 32 and outputs the control signal LRV to the LR motor 33.

The UD motor 32 pulls the UD bending wire based on the control signal UDV and bends the bending portion 21 upward or downward. Due to this, the insertion unit 2 is bent upward or downward. The LR motor 33 pulls the LR bending wire based on the control signal LRV and bends the bending portion 21 leftward or rightward. Due to this, the insertion unit 2 is bent leftward or rightward.

As described above, the control unit 40 calculates the bending control value CO based on the digital value DI1 and the digital value DI2. Furthermore, the control unit may calculate the motor control value MO based on the bending control value CO and may output the motor control value MO to the communication unit 41. The communication unit 41 may transmit the motor control value MO to the communication unit 38. The communication unit 38 may output the motor control value MO to the control unit 30. The control unit 30 may output the motor control value MO to the bending control unit 34.

The control unit 40 may output the digital value DI1 and the digital value DI2 to the communication unit 41. The communication unit 41 may transmit the digital value DI1 and the digital value DI2 to the communication unit 38. The communication unit 38 may output the digital value DI1 and the digital value DI2 to the control unit 30. The control unit 30 may calculate the bending control value CO based on the digital value DI1 and the digital value DI2 and may calculate the motor control value MO based on the bending control value CO. The control unit 30 may output the motor control value MO to the bending control unit 34.

The operation device 4 does not need to include the control unit 40. For example, the main body unit 3 and the operation device 4 may be connected by a cable, and the digital value DI1 and the digital value DI2 may be directly output from the ADC 43 and the ADC 45, respectively, to the main body unit 3.

In the following descriptions, there is a case in which a tilted state of the housing 48 is used instead of the rotation amount of the housing 48. For example, the tilted state of the housing 48 is indicated by an angle by which the operation device 4 is tilted. The angle is equivalent to the rotation amount of the housing 48.

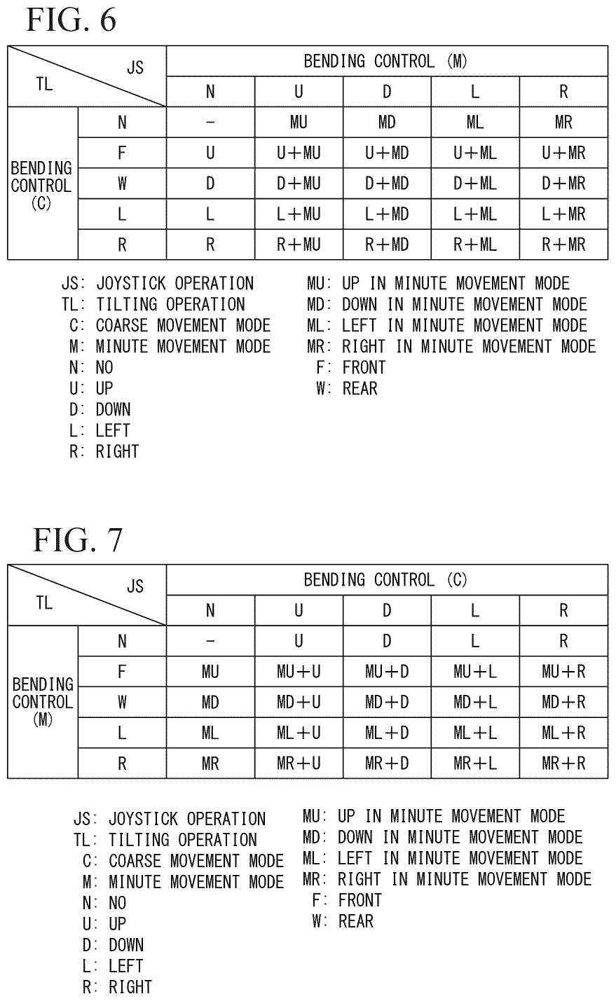

The bending control in the first embodiment will be described below by using FIGS. 6 to 11. FIGS. 6 to 10 show examples of a relationship between operations performed by a user and details of bending control.

FIG. 6 shows a first example of the bending control in accordance with a combination of the joystick operation (JS) and the tilting operation (TL). The joystick operation is associated with the bending control (first control) in the minute movement mode (M). The tilting operation is associated with the bending control (second control) in the coarse movement mode (C).

A bending amount in the coarse movement mode changes in accordance with an angle by which the operation device 4 is tilted. The bending amount increases as the angle increases. A bending amount in the minute movement mode changes in accordance with an angle by which the joystick 42 is tilted. The bending amount increases as the angle increases.

A maximum bending amount in the coarse movement mode is larger than a maximum bending amount in the minute movement mode. For example, when a user tilts the operation device 4 to a maximum angle, the maximum bending amount in the coarse movement mode is Bmax1. For example, when the user tilts the joystick 42 to the maximum angle, the maximum bending amount in the minute movement mode is Bmax2. The bending amount Bmax1 is larger than the bending amount Bmax2.

When a user performs the tilting operation without performing the joystick operation, the bending direction corresponds to the direction in which the user tilts the operation device 4. For example, when the user tilts the operation device 4 in the left direction L1 shown in FIG. 2, the control unit 40 calculates a bending control value (second control value) used for bending the insertion unit 2 leftward in accordance with the tilting operation. The insertion unit 2 is bent leftward by a bending amount in the coarse movement mode.

On the other hand, when the user performs the joystick operation without performing the tilting operation, the bending direction corresponds to the direction in which the user tilts the joystick 42. For example, when the user tilts the joystick 42 in the left direction L2 shown in FIG. 4, the control unit 40 calculates a bending control value (first control value) used for bending the insertion unit 2 leftward in accordance with the joystick operation. The insertion unit 2 is bent leftward by a bending amount in the minute movement mode.

When the user performs the tilting operation and the joystick operation, the bending direction corresponds to the direction in which the user tilts the operation device 4 and the direction in which the user tilts the joystick 42. For example, the user tilts the operation device 4 in the left direction L1 shown in FIG. 2 and tilts the joystick 42 in the upward direction U2 shown in FIG. 4. The control unit 40 calculates a bending control value (second control value) used for bending the insertion unit 2 leftward in accordance with the tilting operation and calculates a bending control value (first control value) used for bending the insertion unit 2 upward in accordance with the joystick operation. The insertion unit 2 is bent leftward by a bending amount in the coarse movement mode and is bent upward by a bending amount in the minute movement mode.

The user can set an operation mode of the endoscope device 10 to the normal mode or the fine mode by operating the touch panel 36, the operation button 37, or the operation button 46. The normal mode and the fine mode are not simultaneously set in the endoscope device 10, and only the normal mode or the fine mode is set in the endoscope device 10. The user can switch the operation modes of the endoscope device between the normal mode and the fine mode.

When the normal mode is set in the endoscope device 10, the endoscope system 1 may execute the first control and the second control in accordance with the relationship shown in FIG. 6. Alternatively, when the fine mode is set in the endoscope device 10, the endoscope system 1 may execute the first control and the second control in accordance with the relationship shown in FIG. 6.

When the normal mode or the fine mode is set in the endoscope device 10 and the user performs neither the tilting operation nor the joystick operation, the insertion unit 2 is not bent. At this time, the bending amount of the insertion unit 2 is 0, and the position of the distal end portion 2a is an initial position. When the normal mode is set in the endoscope device 10 and the user performs the tilting operation or the joystick operation, the insertion unit 2 is bent in accordance with the tilting operation or the joystick operation. When the user stops the tilting operation or the joystick operation, the insertion unit 2 stops being bent. At this time, the distal end portion 2a returns to the initial position.

When the fine mode is set in the endoscope device 10 and the user performs the tilting operation or the joystick operation, the insertion unit 2 is bent. When the user stops the tilting operation or the joystick operation, the insertion unit 2 stops being bent. At this time, the insertion unit 2 keeps being bent. The position of the distal end portion 2a is different from the initial position. After the user restarts the tilting operation or the joystick operation, the insertion unit 2 is bent in accordance with the tilting operation or the joystick operation. The distal end portion 2a moves from the position different from the initial position.

FIG. 7 shows a second example of the bending control in accordance with a combination of the joystick operation (JS) and the tilting operation (TL). The joystick operation is associated with the bending control (first control) in the coarse movement mode (C). The tilting operation is associated with the bending control (second control) in the minute movement mode (M).

When a user performs the tilting operation without performing the joystick operation, the bending direction corresponds to the direction in which the user tilts the operation device 4. For example, when the user tilts the operation device 4 in the left direction L1 shown in FIG. 2, the control unit 40 calculates a bending control value (second control value) used for bending the insertion unit 2 leftward in accordance with the tilting operation. The insertion unit 2 is bent leftward by a bending amount in the minute movement mode.

On the other hand, when the user performs the joystick operation without performing the tilting operation, the bending direction corresponds to the direction in which the user tilts the joystick 42. For example, when the user tilts the joystick 42 in the left direction L2 shown in FIG. 4, the control unit 40 calculates a bending control value (first control value) used for bending the insertion unit 2 leftward in accordance with the joystick operation. The insertion unit 2 is bent leftward by a bending amount in the coarse movement mode.

When the user performs the tilting operation and the joystick operation, the bending direction corresponds to the direction in which the user tilts the operation device 4 and the direction in which the user tilts the joystick 42. For example, the user tilts the operation device 4 in the left direction L1 shown in FIG. 2 and tilts the joystick 42 in the upward direction U2 shown in FIG. 4. The control unit 40 calculates a bending control value (second control value) used for bending the insertion unit 2 leftward in accordance with the tilting operation and calculates a bending control value (first control value) used for bending the insertion unit 2 upward in accordance with the joystick operation. The insertion unit 2 is bent leftward by a bending amount in the minute movement mode and is bent upward by a bending amount in the coarse movement mode.

When the normal mode is set in the endoscope device 10, the endoscope system 1 may execute the first control and the second control in accordance with the relationship shown in FIG. 7. Alternatively, when the fine mode is set in the endoscope device 10, the endoscope system 1 may execute the first control and the second control in accordance with the relationship shown in FIG. 7.

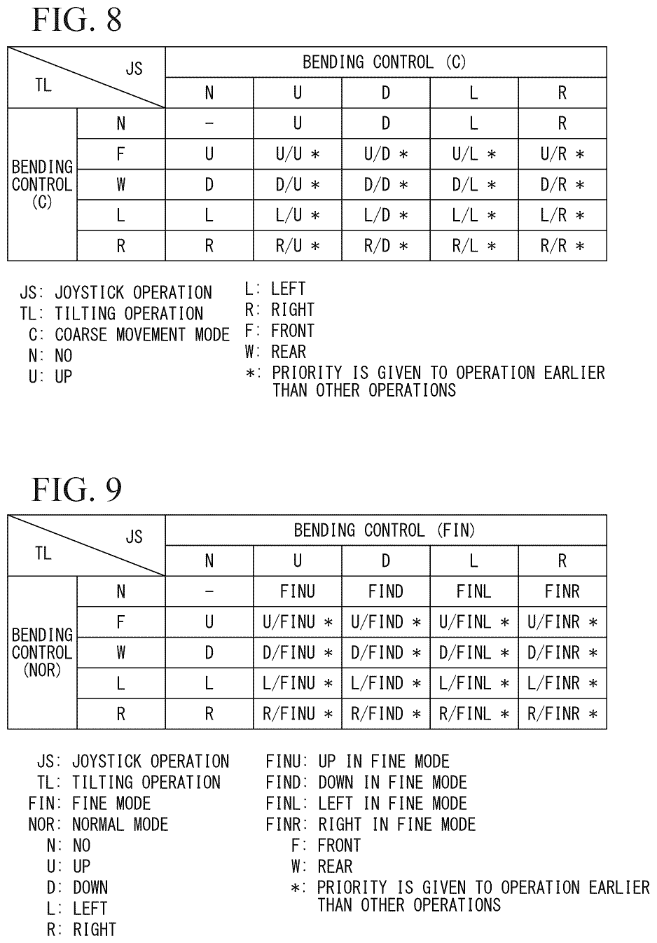

FIG. 8 shows a third example of the bending control in accordance with a combination of the joystick operation (JS) and the tilting operation (TL). The joystick operation is associated with the bending control (first control) in the coarse movement mode (C). The tilting operation is associated with the bending control (second control) in the coarse movement mode (C).

When a user performs the tilting operation without performing the joystick operation, the bending direction corresponds to the direction in which the user tilts the operation device 4. The control unit 40 calculates a bending control value (second control value) used for bending the insertion unit 2 in accordance with the tilting operation. The insertion unit 2 is bent by a bending amount in the coarse movement mode.

On the other hand, when the user performs the joystick operation without performing the tilting operation, the bending direction corresponds to the direction in which the user tilts the joystick 42. The control unit 40 calculates a bending control value (first control value) used for bending the insertion unit 2 in accordance with the joystick operation. The insertion unit 2 is bent by a bending amount in the coarse movement mode.

When the user performs the tilting operation and the joystick operation, the bending direction corresponds to the direction in which the user tilts the operation device 4 and the direction in which the user tilts the joystick 42. In a case in which the tilting operation is started prior to the joystick operation, the tilting operation is prioritized over the joystick operation. While the user performs only the tilting operation, the control unit 40 calculates a bending control value (second control value) used for bending the insertion unit 2 in accordance with the tilting operation. The insertion unit 2 is bent by a bending amount in the coarse movement mode.

Thereafter, the user starts the joystick operation and performs the tilting operation and the joystick operation at the same time. In a predetermined period after the user has started the joystick operation, the control unit 40 calculates a bending control value (second control value) used for bending the insertion unit 2 in accordance with the tilting operation. The insertion unit 2 is bent by a bending amount in the coarse movement mode. The results of the joystick operation are invalid in the predetermined period.

After the predetermined period, the control unit 40 calculates a bending control value (second control value) used for bending the insertion unit 2 in accordance with the tilting operation and calculates a bending control value (first control value) used for bending the insertion unit 2 in accordance with the joystick operation. The insertion unit 2 is bent by a bending amount in the coarse movement mode. At this time, a bending amount calculated in accordance with the joystick operation is added to a bending amount calculated in accordance with the tilting operation. The insertion unit 2 may be bent by a bending amount in the coarse movement mode in accordance with the tilting operation and the joystick operation from a timing at which the user has started the joystick operation.

On the other hand, in a case in which the joystick operation is started prior to the tilting operation, the joystick operation is prioritized over the tilting operation. While the user performs only the joystick operation, the control unit 40 calculates a bending control value (first control value) used for bending the insertion unit 2 in accordance with the joystick operation. The insertion unit 2 is bent by a bending amount in the coarse movement mode.

Thereafter, the user starts the tilting operation and performs the tilting operation and the joystick operation at the same time. In a predetermined period after the user has started the tilting operation, the control unit 40 calculates a bending control value (first control value) used for bending the insertion unit 2 in accordance with the joystick operation. The insertion unit 2 is bent by a bending amount in the coarse movement mode. The results of the tilting operation are invalid in the predetermined period.

After the predetermined period, the control unit 40 calculates a bending control value (second control value) used for bending the insertion unit 2 in accordance with the tilting operation and calculates a bending control value (first control value) used for bending the insertion unit 2 in accordance with the joystick operation. The insertion unit 2 is bent by the sum of the bending amount calculated in accordance with the tilting operation and the bending amount calculated in accordance with the joystick operation. The insertion unit 2 may be bent by a bending amount in the coarse movement mode in accordance with the tilting operation and the joystick operation from a timing at which the user has started the tilting operation.

When the normal mode is set in the endoscope device 10, the endoscope system 1 may execute the first control and the second control in accordance with the relationship shown in FIG. 8. Alternatively, when the fine mode is set in the endoscope device 10, the endoscope system 1 may execute the first control and the second control in accordance with the relationship shown in FIG. 8.

FIG. 9 shows a fourth example of the bending control in accordance with a combination of the joystick operation (JS) and the tilting operation (TL). The joystick operation is associated with the bending control (first control) in the fine mode (FIN). The tilting operation is associated with the bending control (second control) in the normal mode (NOR).

When a user performs the tilting operation without performing the joystick operation, the bending direction corresponds to the direction in which the user tilts the operation device 4. The control unit 40 calculates a bending control value (second control value) used for bending the insertion unit 2 in accordance with the tilting operation. The insertion unit 2 is bent by a bending amount in the normal mode.

On the other hand, when the user performs the joystick operation without performing the tilting operation, the bending direction corresponds to the direction in which the user tilts the joystick 42. The control unit 40 calculates a bending control value (first control value) used for bending the insertion unit 2 in accordance with the joystick operation. The insertion unit 2 is bent by a bending amount in the fine mode.

When the user performs the tilting operation and the joystick operation, the bending direction corresponds to the direction in which the user tilts the operation device 4 and the direction in which the user tilts the joystick 42. In a case in which the tilting operation is started prior to the joystick operation, the tilting operation is prioritized over the joystick operation. While the user performs only the tilting operation, the control unit 40 calculates a bending control value (second control value) used for bending the insertion unit 2 in accordance with the tilting operation. The insertion unit 2 is bent by a bending amount in the normal mode.

Thereafter, the user starts the joystick operation and performs the tilting operation and the joystick operation at the same time. While the user performs the tilting operation and the joystick operation at the same time, the control unit 40 calculates a bending control value (second control value) used for bending the insertion unit 2 in accordance with the tilting operation. The insertion unit 2 is bent by a bending amount in the normal mode. At this time, the results of the joystick operation are invalid.

Thereafter, the user completes the tilting operation and performs only the joystick operation. When the user completes the tilting operation, the operation mode of the endoscope device 10 is changed from the normal mode to the fine mode. While the user performs the joystick operation, the control unit 40 calculates a bending control value (first control value) used for bending the insertion unit 2 in accordance with the joystick operation. The insertion unit 2 is bent by a bending amount in the fine mode.

On the other hand, in a case in which the joystick operation is started prior to the tilting operation, the joystick operation is prioritized over the tilting operation. While the user performs only the joystick operation, the control unit 40 calculates a bending control value (first control value) used for bending the insertion unit 2 in accordance with the joystick operation. The insertion unit 2 is bent by a bending amount in the fine mode.

Thereafter, the user starts the tilting operation and performs the tilting operation and the joystick operation at the same time. While the user performs the tilting operation and the joystick operation at the same time, the control unit 40 calculates a bending control value (first control value) used for bending the insertion unit 2 in accordance with the joystick operation. The insertion unit 2 is bent by a bending amount in the fine mode. At this time, the results of the tilting operation are invalid.

Thereafter, the user completes the joystick operation and performs only the tilting operation. When the user completes the joystick operation, the operation mode of the endoscope device 10 is changed from the fine mode to the normal mode. While the user performs the tilting operation, the control unit 40 calculates a bending control value (second control value) used for bending the insertion unit 2 in accordance with the tilting operation. The insertion unit 2 is bent by a bending amount in the normal mode.

In a case in which the joystick operation is started prior to the tilting operation, the endoscope system 1 may execute the following control. As described above, while the user performs the tilting operation and the joystick operation at the same time, the control unit 40 calculates a bending control value (first control value) used for bending the insertion unit 2 in accordance with the joystick operation. The insertion unit 2 is bent by a bending amount in the fine mode.

When the user releases the joystick 42 from his/her hand, the joystick 42 returns to the center position quickly. At this time, the operation mode of the endoscope device is changed from the fine mode to the normal mode. In addition, a bending amount calculated in accordance with the tilting operation returns to 0 slowly. While the user performs the tilting operation, the control unit 40 calculates a bending control value (second control value) used for bending the insertion unit 2 in accordance with the tilting operation. The insertion unit 2 is bent by a bending amount in the normal mode.

The joystick operation may be associated with the bending control (first control) in the normal mode (NOR), and the tilting operation may be associated with the bending control (second control) in the fine mode (FIN).

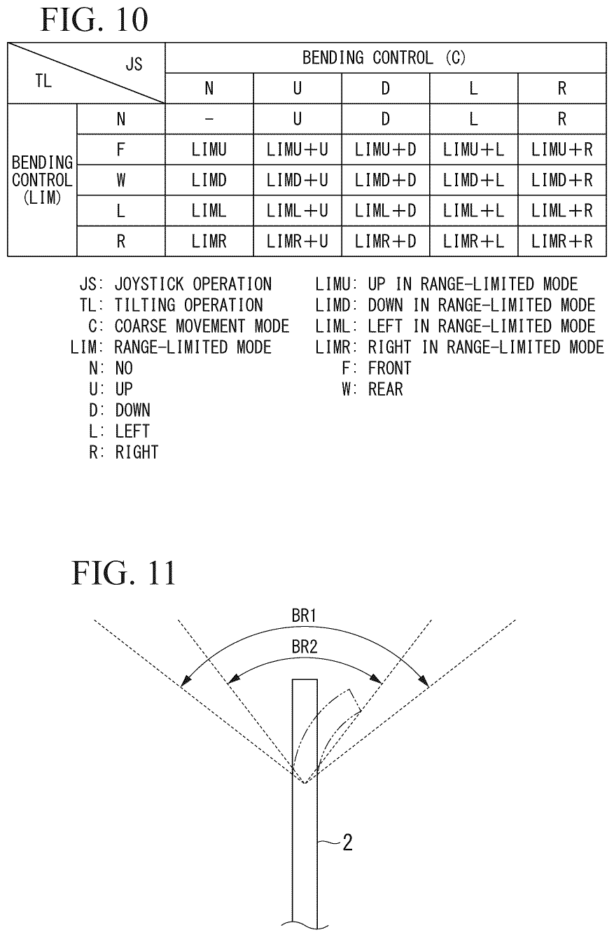

FIG. 10 shows a fifth example of the bending control in accordance with a combination of the joystick operation (JS) and the tilting operation (TL). The joystick operation is associated with the bending control (first control) in the coarse movement mode (C). The tilting operation is associated with the bending control (second control) in a range-limited mode (LIM).

FIG. 11 shows a range (bending range) in which the insertion unit 2 can be bent. The bending range indicates positions which the insertion unit 2 reachs through the bending control executed once or more. The insertion unit 2 can be bent in a bending range BR1 in the coarse movement mode, the minute movement mode, the normal mode, or the fine mode. On the other hand, the insertion unit 2 can be bent in a bending range BR2 in the range-limited mode. The bending range BR2 is narrower than the bending range BR1.

A bending amount in the range-limited mode changes in accordance with an angle by which the operation device 4 is tilted. The bending amount increases as the angle increases. A bending amount in the coarse movement mode changes in accordance with an angle by which the joystick 42 is tilted. The bending amount increases as the angle increases.

When a user performs the tilting operation without performing the joystick operation, the bending direction corresponds to the direction in which the user tilts the operation device 4. The control unit 40 calculates a bending control value (second control value) used for bending the insertion unit 2 in accordance with the tilting operation. The insertion unit 2 is bent by a bending amount in the range-limited mode.

On the other hand, when the user performs the joystick operation without performing the tilting operation, the bending direction corresponds to the direction in which the user tilts the joystick 42. The control unit 40 calculates a bending control value (first control value) used for bending the insertion unit 2 in accordance with the joystick operation. The insertion unit 2 is bent by a bending amount in the coarse movement mode.

When the user performs the tilting operation and the joystick operation, the bending direction corresponds to the direction in which the user tilts the operation device 4 and the direction in which the user tilts the joystick 42. For example, the user tilts the operation device 4 in the left direction L1 shown in FIG. 2 and tilts the joystick 42 in the upward direction U2 shown in FIG. 4. The control unit 40 calculates a bending control value (second control value) used for bending the insertion unit 2 leftward in accordance with the tilting operation and calculates a bending control value (first control value) used for bending the insertion unit 2 upward in accordance with the joystick operation. The insertion unit 2 is bent leftward by a bending amount in the range-limited mode and is bent upward by a bending amount in the coarse movement mode.

The joystick operation may be associated with the bending control (first control) in the range-limited mode (LIM), and the tilting operation may be associated with the bending control (second control) in the coarse movement mode (C).

When the normal mode is set in the endoscope device 10, the endoscope system 1 may execute the first control and the second control n accordance with the relationship shown in FIG. 10. Alternatively, when the fine mode is set in the endoscope device 10, the endoscope system 1 may execute the first control and the second control n accordance with the relationship shown in FIG. 10.

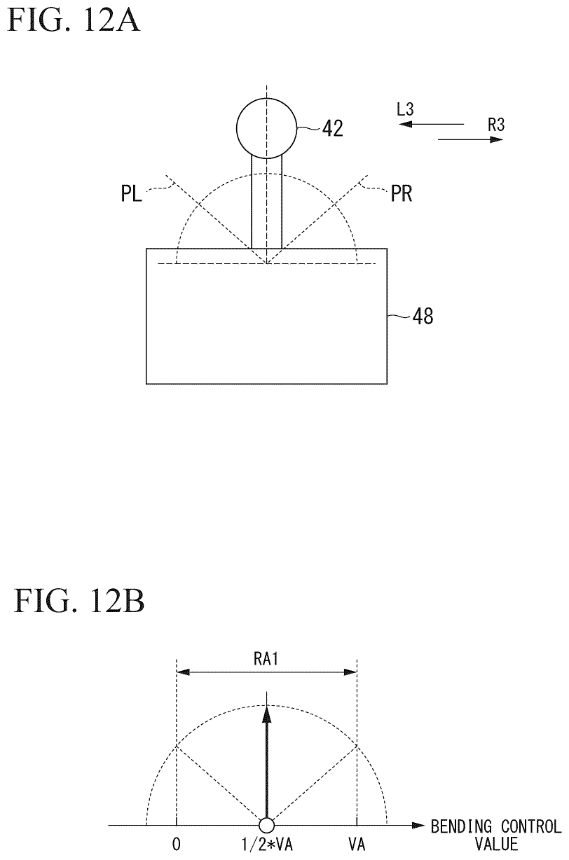

Examples of a bending control value in the joystick operation and the tilting operation will be described by using FIGS. 12A to 25. FIG. 12A and the like show examples of the state of the joystick 42 and the posture of the housing 48. FIG. 12B and the like show examples of a bending control value. Bending control values shown in FIG. 12B and the like correspond to the bending control value CO calculated by the control unit 40.

Hereinafter, a method in which the control unit 40 calculates a bending control value when a user tilts the joystick 42 in the left direction L2 or the right direction R2 shown in FIG. 4 will be described. When the user tilts the joystick 42 in the upward direction U2 or the downward direction D2 shown in FIG. 4, the control unit 40 can calculate a bending control value by using a similar method to that described below.

Hereinafter, a method in which the control unit 40 calculates a bending control value when a user tilts the operation device 4 in the left direction L1 or the right direction R1 shown in FIG. 2 will be described. When the user tilts the operation device 4 in the front direction F1 or the rear direction W1 shown in FIG. 3, the control unit 40 can calculate a bending control value by using a similar method to that described below.

Three or more different control values can be used as bending control values. The three or more control values correspond to three or more different bending amounts of the insertion unit 2. One control value corresponds to one bending amount. The control unit 40 selects one of the three or more control values. The memory 47 may store a look-up table including the three or more control values. The control unit 40 may select one control value from the look-up table.

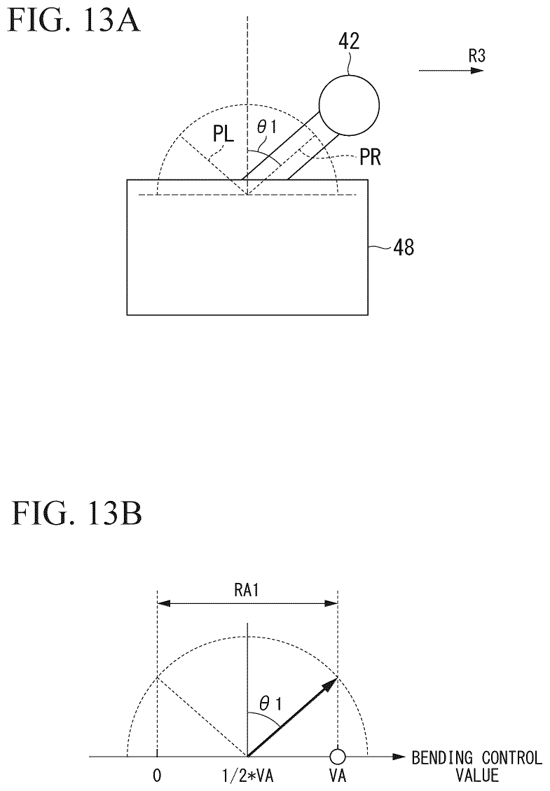

FIG. 12A, FIG. 12B, FIG. 13A, and FIG. 13B show examples of a bending control value calculated in accordance with the joystick operation in the coarse movement mode. When a user is not operating the joystick 42, the joystick 42 is almost perpendicular to the surface of the housing 48 as shown in FIG. 12A. The user can tilt the joystick 42 to a position PL in a left direction L3 and can tilt the joystick 42 to a position PR in a right direction R3. The position of the joystick 42 is restricted from the position PL to the position PR. The joystick 42 is movable between the position PL and the position PR.

FIG. 12B shows a bending control value corresponding to the angle of the joystick 42 shown in FIG. 12A. The control unit 40 calculates a bending control value in a range RA1 in accordance with the operation of the joystick 42 in the coarse movement mode. The minimum value of the range RA1 is 0, and the maximum value of the range RA1 is VA. When the bending control value is 0, the bending amount of the insertion unit 2 leftward in the coarse movement mode is the maximum. When the bending control value is VA, the bending amount of the insertion unit 2 rightward in the coarse movement mode is the maximum. When the joystick 42 is in the state shown in FIG. 12A, the bending control value (first control value) is ½*VA. At this time, the insertion unit 2 is not bent.

When the user tilts the joystick 42 to the position PR in the right direction R3, the angle of the joystick 42 is θ1 shown in FIG. 13A. The angle θ1 is less than 90 degrees. At this time, the bending control value (first control value) is VA as shown in FIG. 13B. At this time, the insertion unit 2 is bent rightward.

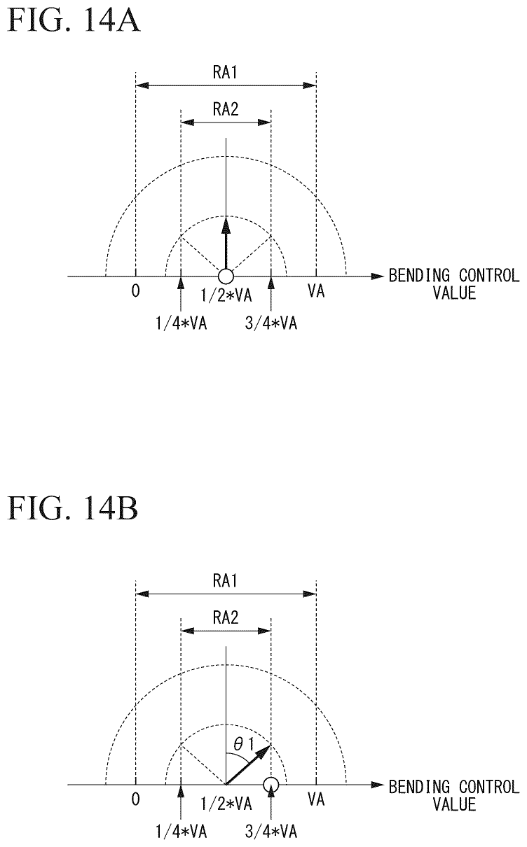

FIG. 14A and FIG. 14B show an example of a bending control value calculated in accordance with the joystick operation in the minute movement mode. When a user is not operating the joystick 42, the sate of the joystick 42 is the same state as that shown in FIG. 12A described above.

FIG. 14A shows a bending control value corresponding to the angle of the joystick 42 shown in FIG. 12A. The control unit 40 calculates a bending control value in a range RA2 in accordance with the operation of the joystick 42 in the minute movement mode. The minimum value of the range RA2 is ¼*VA, and the maximum value of the range RA2 is ¾*VA. When the bending control value is ¼*VA, the bending amount of the insertion unit 2 leftward in the minute movement mode is the maximum. When the bending control value is ¾*VA, the bending amount of the insertion unit 2 rightward in the minute movement mode is the maximum. The range RA2 of bending control values in the minute movement mode is narrower than the range RA1 of bending control values in the coarse movement mode. Therefore, the maximum bending amount in the minute movement mode is less than that in the coarse movement mode. When the joystick 42 is in the state shown in FIG. 12A, the bending control value (first control value) is ½*VA as shown in FIG. 14A. At this time, the insertion unit 2 is not bent.

When the user tilts the joystick 42 to the position PR in the right direction R3, the state of the joystick 42 is the same as that shown in FIG. 13A described above. FIG. 14B shows a bending control value corresponding to the angle of the joystick 42 shown in FIG. 13A. When the joystick 42 is in the state shown in FIG. 13A, the bending control value (first control value) is ¾*VA as shown in FIG. 14B. At this time, the insertion unit 2 is bent rightward.

When the user tilts the joystick 42 to the position PR in the right direction R3, the bending control value in the coarse movement mode is VA shown in FIG. 13B, and the bending control value in the minute movement mode is ¾*VA shown in FIG. 14B. The bending control value in the minute movement mode is less than that in the coarse movement mode. When the angle of the joystick 42 is a predetermined angle, the bending amount in the minute movement mode is less than that in the coarse movement mode.

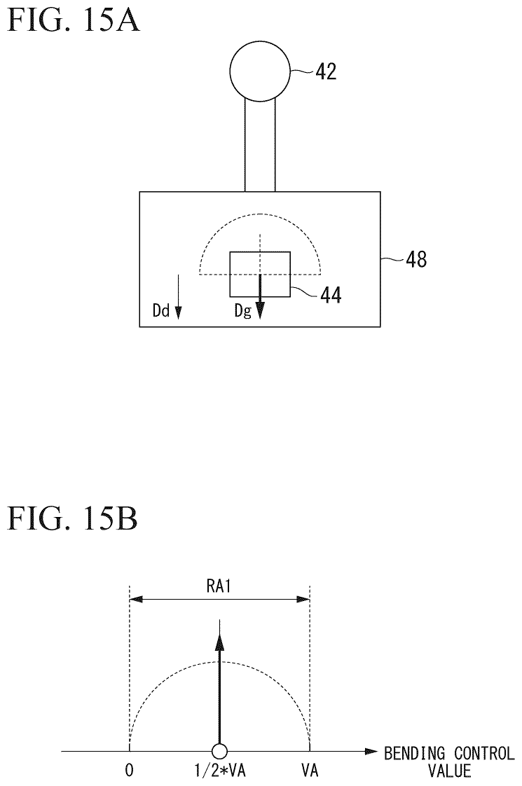

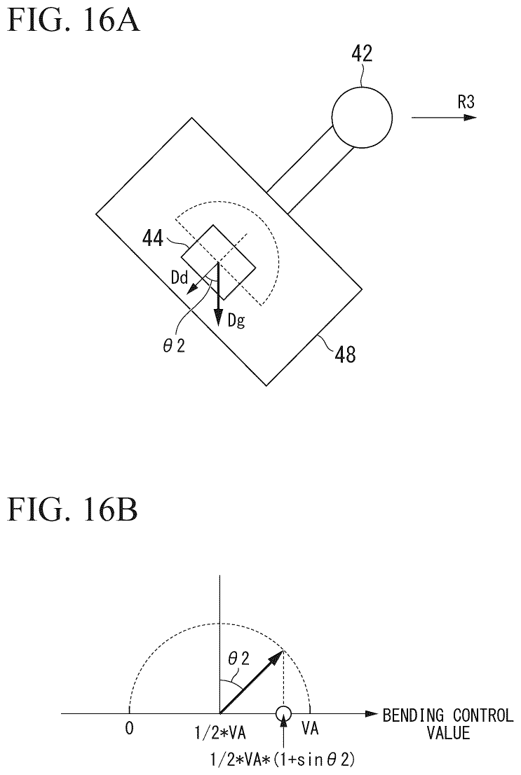

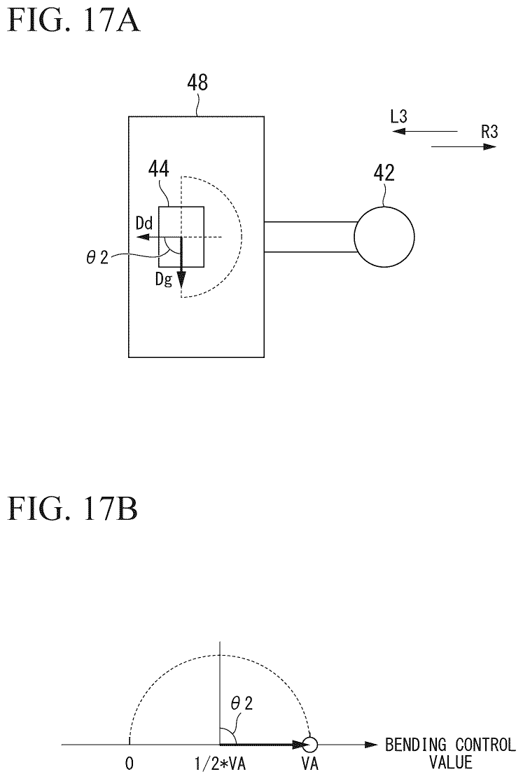

FIG. 15A, FIG. 15B, FIG. 16A, FIG. 16B, FIG. 17A, and FIG. 17B show examples of a bending control value calculated in accordance with the tilting operation in the coarse movement mode.

A user inputs a reset instruction to the endoscope device 10 by operating the operation button 46. The motion sensor 44 determines, as movement of the housing 48, a change amount of the posture of the housing 48 from a timing at which the reset instruction has been input. For example, when a downward direction Dd of the housing 48 is the same as a gravitational direction Dg as shown in FIG. 15A, the user inputs the reset instruction to the endoscope device 10.

FIG. 15B shows a bending control value corresponding to the tilted state of the housing 48 shown in FIG. 15A. The control unit 40 calculates a bending control value in a range RA1 in accordance with the tilted state of the housing 48 in the coarse movement mode. The minimum value of the range RA1 is 0, and the maximum value of the range RA1 is VA. When the bending control value is 0, the bending amount of the insertion unit 2 leftward in the coarse movement mode is the maximum. When the bending control value is VA, the bending amount of the insertion unit 2 rightward in the coarse movement mode is the maximum. When the housing 48 is tilted in the state shown in FIG. 15A, the bending control value (second control value) is ½*VA. At this time, the insertion unit 2 is not bent.

For example, a user tilts the housing 48 in a right direction R3 as shown in FIG. 16A. At this time, an angle θ2 between a downward direction Dd of the housing 48 and a gravitational direction Dg is less than 90 degrees. At this time, the bending control value (second control value) is ½*VA*(1+ sin θ2) as shown in FIG. 16B. At this time, the insertion unit 2 is bent rightward.

For example, a user tilts the housing 48 in a right direction R3 as shown in FIG. 17A. At this time, an angle θ2 between a downward direction Dd of the housing 48 and a gravitational direction Dg is 90 degrees. At this time, the bending control value (second control value) is VA as shown in FIG. 17B. At this time, the insertion unit 2 is bent rightward.

When the angle θ2 is greater than 90 degrees, the bending control value is fixed to VA. In addition, when a user tilts the housing 48 in the left direction L3 and the angle θ2 is greater than 90 degrees, the bending control value is fixed to 0.

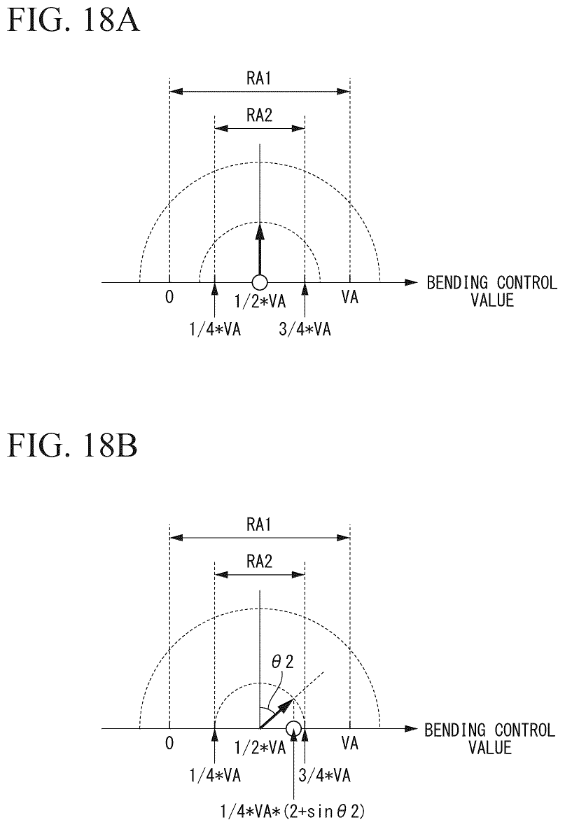

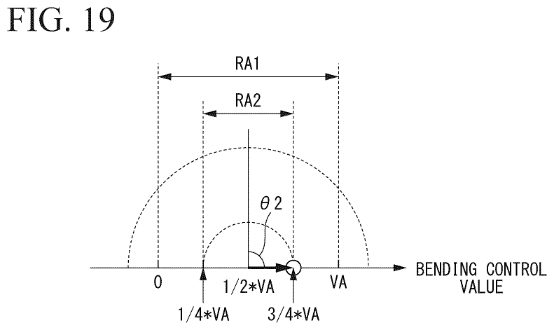

FIG. 18A, FIG. 18B, and FIG. 19 show examples of a bending control value calculated in accordance with the tilting operation in the minute movement mode.

The control unit 40 calculates a bending control value in a range RA2 shown in FIG. 18A in accordance with the tilted state of the housing 48 in the minute movement mode. The minimum value of the range RA2 is ¼*VA, and the maximum value of the range RA2 is ¾*VA. When the bending control value is ¼*VA, the bending amount of the insertion unit 2 leftward in the minute movement mode is the maximum. When the bending control value is ¾*VA, the bending amount of the insertion unit 2 rightward in the minute movement mode is the maximum. The range RA2 of bending control values in the minute movement mode is narrower than the range RA1 of bending control values in the coarse movement mode. When the downward direction Dd of the housing 48 is the same as the gravitational direction Dg as shown in FIG. 15A described above, the bending control value (second control value) is ½*VA as shown in FIG. 18A. At this time, the insertion unit 2 is not bent.

For example, when a user tilts the housing 48 in the right direction R3 as shown in FIG. 16A described above, the angle θ2 between the downward direction Dd of the housing 48 and the gravitational direction Dg is less than 90 degrees. At this time, the bending control value (second control value) is ¼*VA*(2+sin θ2) as shown in FIG. 18B. At this time, the insertion unit 2 is bent rightward.

For example, when a user tilts the housing 48 in the right direction R3 as shown in FIG. 17A described above, the angle θ2 between the downward direction Dd of the housing 48 and the gravitational direction Dg is 90 degrees. At this time, the bending control value (second control value) is ¾*VA as shown in FIG. 19. At this time, the insertion unit 2 is bent rightward.

When the angle θ2 between the downward direction Dd of the housing 48 and the gravitational direction Dg is less than 90 degrees as shown in FIG. 16A, the bending control value in the coarse movement mode is ½*VA*(1+sin θ2) shown in FIG. 16B, and the bending control value in the minute movement mode is ¼*VA*(2+sin θ2) shown in FIG. 18B. When the angle θ2 between the downward direction Dd of the housing 48 and the gravitational direction Dg is 90 degrees as shown in FIG. 17A, the bending control value in the coarse movement mode is VA shown in FIG. 17B, and the bending control value in the minute movement mode is ¾*VA shown in FIG. 19. The bending control value in the minute movement mode is less than that in the coarse movement mode. When the angle θ2 between the downward direction Dd of the housing 48 and the gravitational direction Dg is a predetermined angle, the bending amount in the minute movement mode is less than that in the coarse movement mode.

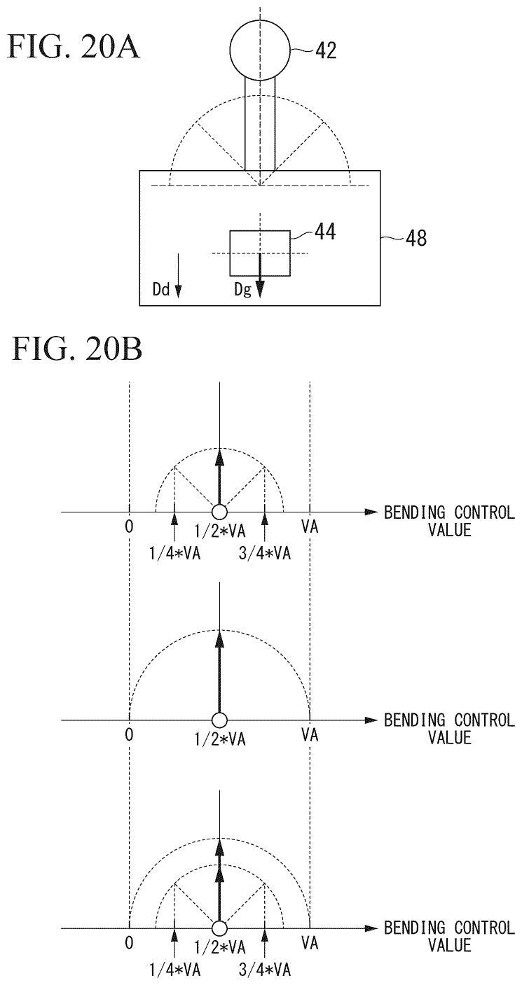

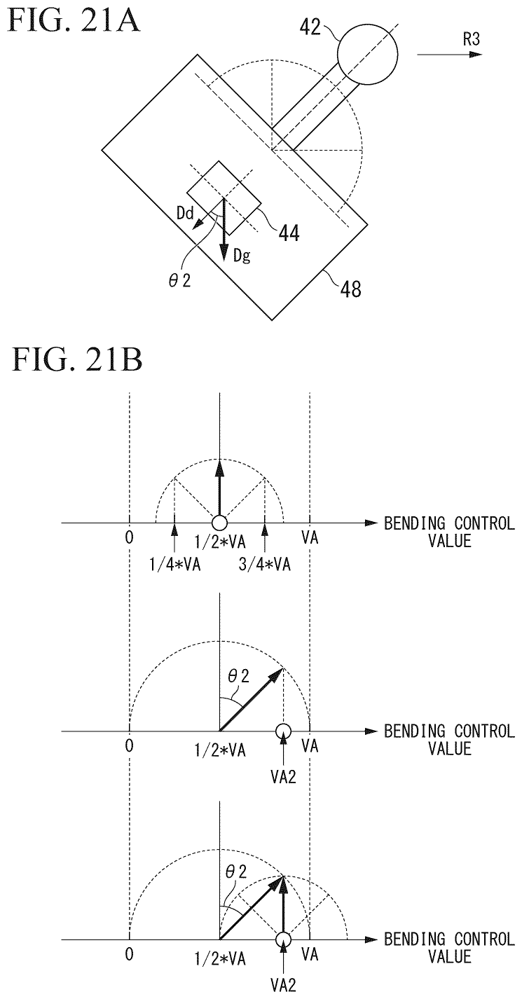

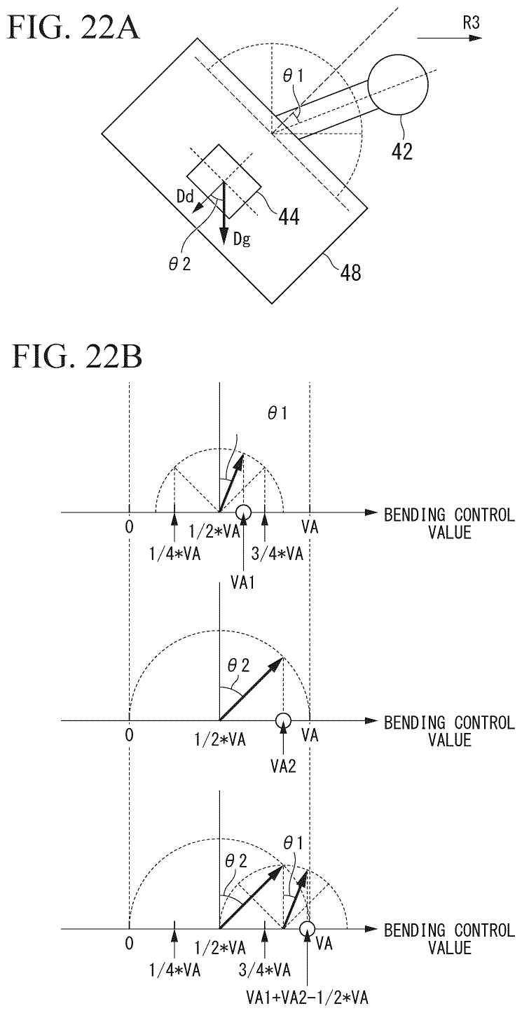

FIG. 20A, FIG. 20B, FIG. 21A, FIG. 21B, FIG. 22A, and FIG. 22B show examples of a bending control value calculated in accordance with the joystick operation in the minute movement mode and the tilting operation in the coarse movement mode.

For example, when a downward direction Dd of the housing 48 is the same as a gravitational direction Dg as shown in FIG. 20A, a user inputs a reset instruction to the endoscope device 10. At this time, the user is not operating the joystick 42.

FIG. 20B shows a bending control value corresponding to the angle of the joystick 42 and the tilted state of the housing 48 shown in FIG. 20A. When the joystick 42 is in the state shown in FIG. 20A, the bending control value (first control value) calculated in accordance with the joystick operation in the minute movement mode is ½*VA. When the housing 48 is in the tilted state shown in FIG. 20A, the bending control value (second control value) calculated in accordance with the tilting operation in the coarse movement mode is ½*VA.

The control unit 40 calculates a final bending control value in light of the bending control value in the minute movement mode and the bending control value in the coarse movement mode. The bending control value is ½*VA. At this time, the insertion unit 2 is not bent.

For example, a user tilts the housing 48 in a right direction R3 as shown in FIG. 21A. At this time, the angle θ2 between a downward direction Dd of the housing 48 and a gravitational direction Dg is less than 90 degrees. At this time, the user is not operating the joystick 42.

FIG. 21B shows a bending control value corresponding to the angle of the joystick 42 and the tilted state of the housing 48 shown in FIG. 21A. When the joystick 42 is in the state shown in FIG. 21A, the bending control value (first control value) calculated in accordance with the joystick operation in the minute movement mode is ½*VA. When the housing 48 is in the tilted state shown in FIG. 21A, the bending control value (second control value) calculated in accordance with the tilting operation in the coarse movement mode is VA2. The bending control value VA2 is ½*VA*(1+sin θ2).

The control unit 40 calculates a final bending control value in light of the bending control value in the minute movement mode and the bending control value in the coarse movement mode. The bending control value is VA2. At this time, the insertion unit 2 is bent rightward.

For example, a user tilts the joystick 42 in a right direction R3 and tilts the housing 48 in the right direction R3 as shown in FIG. 22A. At this time, the angle of the joystick 42 is θ1, and an angle θ2 between a downward direction Dd of the housing 48 and a gravitational direction Dg is less than 90 degrees.

FIG. 22B shows a bending control value corresponding to the angle of the joystick 42 and the tilted state of the housing 48 shown in FIG. 22A. When the joystick 42 is in the state shown in FIG. 22A, the bending control value (first control value) calculated in accordance with the joystick operation in the minute movement mode is VA1. The bending control value VA1 is ¼*VA*(2+sin θ1). When the housing 48 is in the tilted state shown in FIG. 22A, the bending control value (second control value) calculated in accordance with the tilting operation in the coarse movement mode is VA2. The bending control value VA2 is ½*VA*(1+sin θ2).

The control unit 40 calculates a final bending control value in light of the bending control value in the minute movement mode and the bending control value in the coarse movement mode. The bending control value is VA1+VA2−½*VA. At this time, the insertion unit 2 is bent rightward.

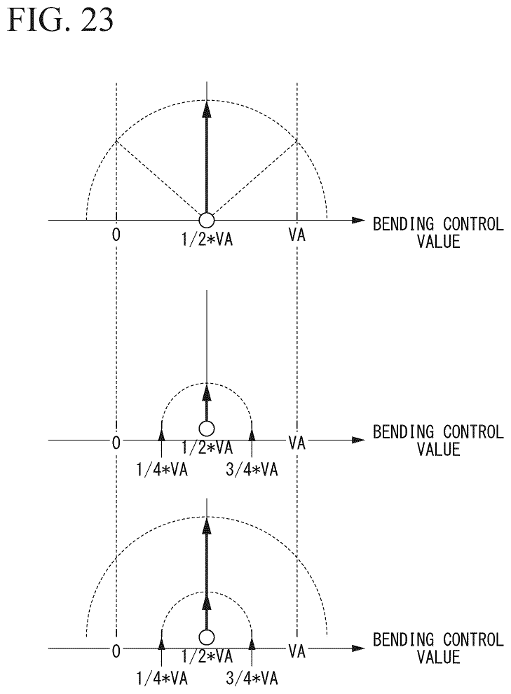

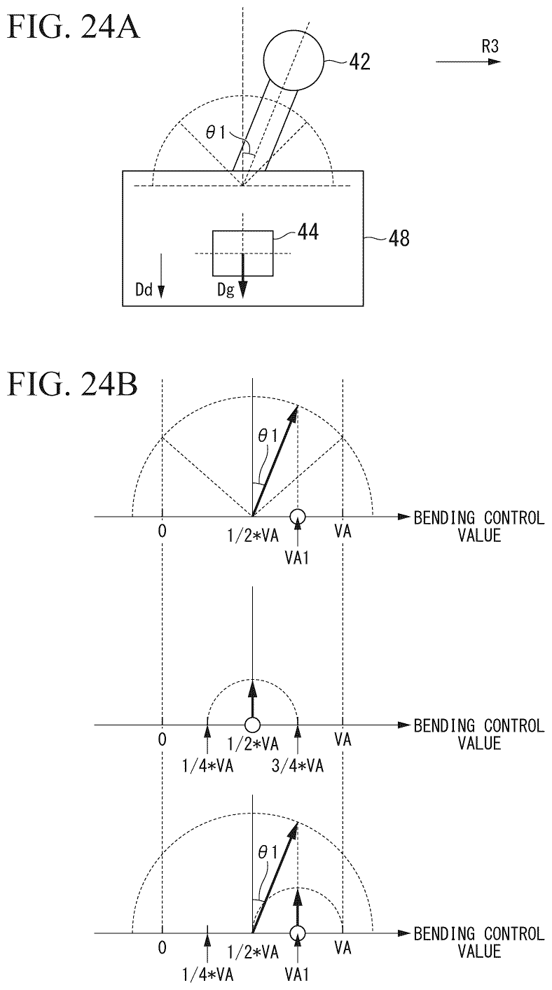

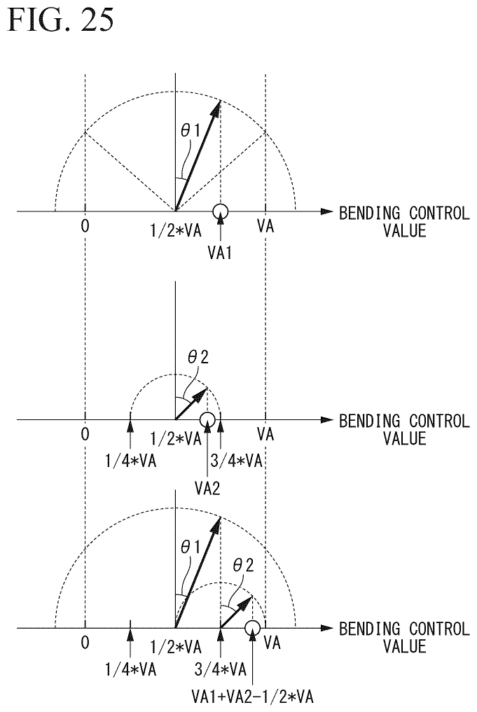

FIG. 23, FIG. 24A, FIG. 24B, and FIG. 25 show examples of a bending control value calculated in accordance with the joystick operation in the coarse movement mode and the tilting operation in the minute movement mode.

For example, when the downward direction Dd of the housing 48 is the same as the gravitational direction Dg as shown in FIG. 20A described above, a user inputs a reset instruction to the endoscope device 10. At this time, the user is not operating the joystick 42.

FIG. 23 shows a bending control value corresponding to the angle of the joystick 42 and the tilted state of the housing 48 shown in FIG. 20A described above. When the joystick 42 is in the state shown in FIG. 20A, the bending control value (first control value) calculated in accordance with the joystick operation in the coarse movement mode is ½*VA. When the housing 48 is in the tilted state shown in FIG. 20A, the bending control value (second control value) calculated in accordance with the tilting operation in the minute movement mode is ½*VA.

The control unit 40 calculates a final bending control value in light of the bending control value in the coarse movement mode and the bending control value in the minute movement mode. The bending control value is ½*VA. At this time, the insertion unit 2 is not bent.

For example, a user tilts the joystick 42 in a right direction R3 as shown in FIG. 24A. At this time, the angle of the joystick 42 is θ1. At this time, the user is not tilting the housing 48, and a downward direction Dd of the housing 48 is the same as a gravitational direction Dg.

FIG. 24B shows a bending control value corresponding to the angle of the joystick 42 and the tilted state of the housing 48 shown in FIG. 24A. When the joystick 42 is in the state shown in FIG. 24A, the bending control value (first control value) calculated in accordance with the joystick operation in the coarse movement mode is VA1. The bending control value VA1 is ½*VA*(1+sin θ1). When the housing 48 is in the tilted state shown in FIG. 24A, the bending control value (second control value) calculated in accordance with the tilting operation in the minute movement mode is ½*VA.

The control unit 40 calculates a final bending control value in light of the bending control value in the coarse movement mode and the bending control value in the minute movement mode. The bending control value is VA1. At this time, the insertion unit 2 is bent rightward.

For example, a user tilts the joystick 42 in the right direction R3 and tilts the housing 48 in the right direction R3 as shown in FIG. 22A described above. At this time, the angle of the joystick 42 is θ1, and the angle θ2 between the downward direction Dd of the housing 48 and the gravitational direction Dg is less than 90 degrees.

FIG. 25 shows a bending control value corresponding to the angle of the joystick 42 and the tilted state of the housing 48 shown in FIG. 22A described above. When the joystick 42 is in the state shown in FIG. 22A, the bending control value (first control value) calculated in accordance with the joystick operation in the coarse movement mode is VA1. The bending control value VA1 is ½*VA*(1+sin θ1). When the housing 48 is in the tilted state shown in FIG. 22A, the bending control value (second control value) calculated in accordance with the tilting operation in the minute movement mode is VA2. The bending control value VA2 is ¼*VA*(2+sin θ2).

The control unit 40 calculates a final bending control value in light of the bending control value in the coarse movement mode and the bending control value in the minute movement mode. The bending control value is VA1+VA2−½*VA. At this time, the insertion unit 2 is bent rightward.

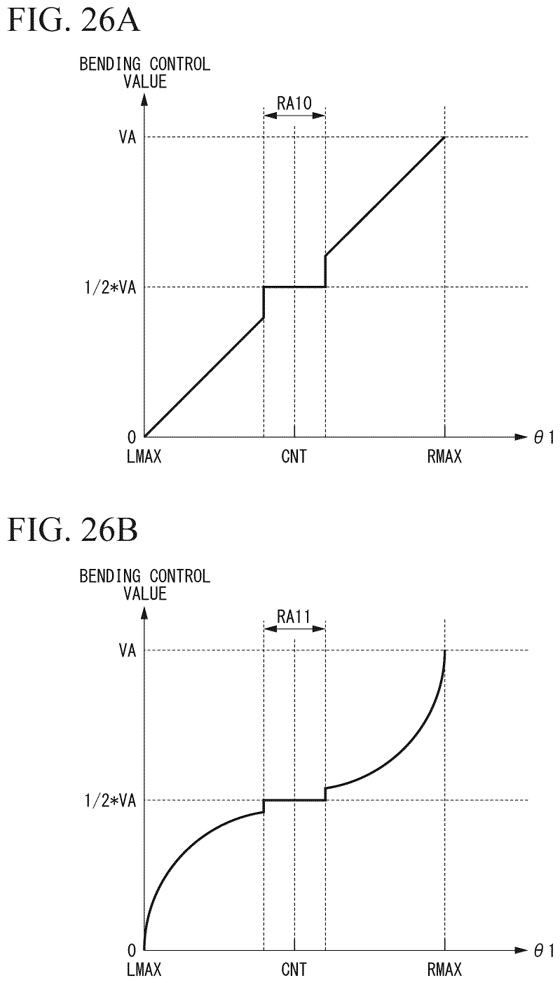

Another example of a bending control value calculated in accordance with the joystick operation will be described. FIG. 26A and FIG. 26B are graphs showing examples of a relationship between an angle of the joystick 42 and a bending control value. The horizontal axis in the graph shown in each drawing indicates an angle (01) of the joystick 42, and the vertical axis in the graph shown in each drawing indicates a bending control value. FIG. 26A and FIG. 26B show a bending control value in the coarse movement mode.

An angle of the joystick 42 ranges from CNT to LMAX or ranges from CNT to RMAX. When the position of the joystick 42 is the position PL shown in FIG. 12A, the angle of the joystick 42 is LMAX. When the position of the joystick 42 is the position PR shown in FIG. 12A described above, the angle of the joystick 42 is RMAX. When a user is not operating the joystick 42, the angle of the joystick 42 is CNT.

FIG. 26A shows a first example. When the angle of the joystick 42 is included in a range RA10 shown in FIG. 26A, the bending control value is a constant value (½*VA). Even when the angle of the joystick 42 changes slightly in the vicinity of CNT, the bending control value is a constant value. Therefore, it is possible to prevent a bending amount of the insertion unit 2 from changing in accordance with the movement of the joystick 42 which is unintended by the user. When the angle of the joystick 42 is not included in the range RA10, the bending control value linearly changes in accordance with the angle of the joystick 42.

FIG. 26B shows a second example. When the angle of the joystick 42 is included in a range RA11 shown in FIG. 26B, the bending control value is a constant value (½*VA). When the angle of the joystick 42 is not included in the range RA11, the bending control value curvilinearly changes in accordance with the angle of the joystick 42.

When a user performs the joystick operation, the control unit 40 calculates a bending control value in the coarse movement mode in accordance with the relationship shown in FIG. 26A or FIG. 26B. When the user performs the joystick operation, the control unit 40 may calculate a bending control value in the minute movement mode in accordance with a similar relationship to that shown in FIG. 26A or FIG. 26B. When the user performs the tilting operation, the control unit 40 may calculate a bending control value in the coarse movement mode or the minute movement mode in accordance with a similar relationship to that relationship shown in FIG. 26A or FIG. 26B.

In the above-described examples, the first control and the second control are the bending control. The first control or the second control may be control different from the bending control. An example in which the first control or the second control is control different from the bending control will be described later in a third embodiment and a sixth embodiment. The first control and the second control may be control different from the bending control. An example in which the first control and the second control are control different from the bending control will be described later in a reference embodiment.

The operation device 4 may include another movable member instead of the joystick 42 as a movable member of the user interface. For example, the operation device 4 may include a button or a switch instead of the joystick 42. The switch may be a slide switch that does not include a rod-shaped movable member or may be a rotary switch. For example, the operation device 4 may include four buttons corresponding to the four directions in which the insertion unit 2 is bent. The control unit 40 may calculate a bending control value in accordance with the amount by which each button is pressed or the length of time during which each button is pressed. For example, the operation device 4 may include four switches corresponding to the four directions in which the insertion unit 2 is bent. The control unit 40 may calculate a bending control value in accordance with a movement amount or a rotation amount of each switch.

The operation device 4 may include a display and a touch panel disposed on the display instead of the joystick 42. The display may display an icon such as a button. A user may move the icon by moving the finger on the touch panel. The control unit 40 may calculate a bending control value in accordance with a movement amount of the finger. The icon does not need to be displayed, and the user may move the finger in any direction on the touch panel. The touch panel may be a touch pad or a track pad.

An endoscope system according to each aspect of the present invention includes the endoscope device 10 including the bendable insertion unit 2 and includes the operation device 4 and the control unit 40. The control unit 40 may be the control unit 30. Alternatively, the control unit 30 and the control unit 40 may realize the following functions. The operation device 4 includes the housing 48, the joystick 42, and the motion sensor 44. The joystick 42 is a user interface disposed on the housing 48. The state of the joystick 42 changes when touching an object such as a user's finger. The joystick 42 generates the analog voltage AN1 (first signal) in accordance with the state of the joystick 42. The motion sensor 44 generates the analog voltage AN2 (second signal) in accordance with physical movement of the operation device 4. The control unit 40 calculates a first control value used in first control of the endoscope device 10 based on the digital value DI1 corresponding to the analog voltage AN1. The control unit 40 calculates a second control value used in second control of the endoscope device 10 based on the digital value DI2 corresponding to the analog voltage AN2. At least one of the first control and the second control is executed to bend the insertion unit 2.

An operation device according to each aspect of the present invention includes the housing 48, the joystick 42, and the motion sensor 44. The joystick 42 generates the analog voltage AN1 (first signal) in accordance with the state of the joystick 42. The motion sensor 44 generates the analog voltage AN2 (second signal) in accordance with physical movement of the operation device 4. The digital value DI1 corresponding to the analog voltage AN1 is used for calculating a first control value used in the first control of the endoscope device 10. The digital value DI2 corresponding to the analog voltage AN2 is used for calculating a second control value used in the second control of the endoscope device 10. At least one of the first control and the second control is executed to bend the insertion unit 2.

Each aspect of the present invention may include the following modified example. The first control and the second control are executed to bend the insertion unit 2.

Each aspect of the present invention may include the following modified example. The maximum value of a bending amount of the insertion unit 2 in the second control is different from the maximum value of a bending amount of the insertion unit 2 in the first control. For example, the maximum value of a bending amount in the coarse movement mode is greater than that of a bending amount in the minute movement mode.

Each aspect of the present invention may include the following modified example. The control unit 40 calculates the first control value in a first period during which the analog voltage AN1 (first signal) is generated without the analog voltage AN2 (second signal) being generated. The control unit 40 calculates the first control value without calculating the second control value in a second period during which the analog voltage AN1 and the analog voltage AN2 are generated after the first period.

For example, a user performs only the joystick operation in the first period. The control unit 40 calculates the first control value in accordance with the joystick operation in the first period. Thereafter, the user performs the joystick operation and the tilting operation in the second period. The control unit 40 calculates the first control value in accordance with the joystick operation in the second period. The control unit may calculate the first control value and the second control value in accordance with the joystick operation and the tilting operation in a third period during which generation of the analog voltage AN1 and the analog voltage AN2 continues after the second period.

Each aspect of the present invention may include the following modified example. The control unit 40 calculates the second control value in a first period during which the analog voltage AN2 (second signal) is generated without the analog voltage AN1 (first signal) being generated. The control unit 40 calculates the second control value without calculating the first control value in a second period during which the analog voltage AN1 and the analog voltage AN2 are generated after the first period.

For example, a user performs only the tilting operation in the first period. The control unit 40 calculates the second control value in accordance with the tilting operation in the first period. Thereafter, the user performs the joystick operation and the tilting operation in the second period. The control unit 40 calculates the second control value in accordance with the tilting operation in the second period. The control unit 40 may calculate the first control value and the second control value in accordance with the joystick operation and the tilting operation in a third period during which generation of the analog voltage AN1 and the analog voltage AN2 continues after the second period.

Each aspect of the present invention may include the following modified example. A range of a bending amount of the insertion unit 2 in the second control is different from that of a bending amount of the insertion unit 2 in the first control. For example, the bending range BR1 in the coarse movement mode, the minute movement mode, the normal mode, or the fine mode is different from the bending range BR2 in the range-limited mode as shown in FIG. 11.

Each aspect of the present invention may include the following modified example. The physical movement of the operation device 4 contains rotation of the operation device 4.

Each aspect of the present invention may include the following modified example. The operation device 4 has an elongated shape. The motion sensor 44 generates the analog voltage AN2 (second signal) n accordance with the rotation of the operation device 4 around the axis AX2 and the axis AX3 which are perpendicular to the longitudinal direction of the operation device 4. The second control is executed to bend the insertion unit 2.

Each aspect of the present invention may include the following modified example. The user interface includes a movable member disposed on the housing 48.

Each aspect of the present invention may include the following modified example. The operation device 4 includes the control unit 30 and the control unit 40.

Each aspect of the present invention may include the following modified example. The endoscope device 10 includes the control unit 30 and the control unit 40.

Each aspect of the present invention may include the following modified example. The control unit 40 selects one of three or more control values corresponding to three or more bending amounts of the insertion unit 2 as the first control value based on the analog voltage AN1 (first signal).

In the first embodiment, the joystick 42 generates the analog voltage AN1 in accordance with the state of the joystick 42. The control unit 40 calculates the first control value used for the first control of the endoscope device 10 based on the digital value DI1 corresponding to the analog voltage AN1. Therefore, the endoscope system 1 can facilitate fine adjustment of control values. In addition, the user can intuitively bend the insertion unit 2 by tilting the operation device 4. A user can bend the insertion unit 2 and can finely adjust a bending amount of the insertion unit 2 by tilting the joystick 42.

### Second Embodiment

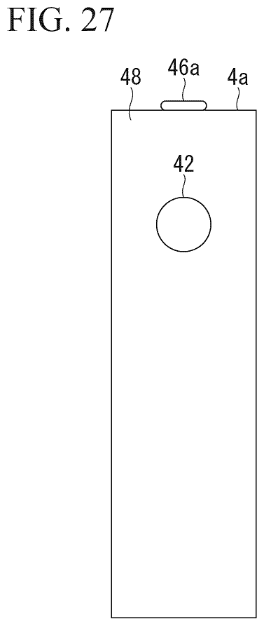

A second embodiment of the present invention will be described. In the second embodiment, the endoscope system 1 shown in FIG. 1 is used. The operation device 4 shown in FIGS. 2 to 4 is changed to an operation device 4a shown in FIG. 27. FIG. 27 shows an external appearance of the operation device 4a.

The operation device 4a includes the joystick 42 and the like shown in FIG. 1 similarly to the operation device 4. The operation device 4a includes a housing 48. The motion sensor 44 and the like shown in FIG. 1 are disposed in the housing 48. The operation device 4a includes an operation button 46a. The operation button 46a is included in the operation button 46 shown in FIG. 1. The operation button 46a is disposed on the housing 48.

In the example shown in FIG. 27, the operation button 46a is disposed on an upper side surface of the housing 48. The position of the operation button 46a is not limited to that shown in FIG. 27. For example, the operation button 46a may be disposed on the front surface or the reverse surface of the housing 48.

The operation button 46a functions as a switch that switches between a first state and a second state. For example, the first state is a state in which the operation button 46a is pressed, and the second state is a state in which the operation button 46a is not pressed. While a user keeps pressing the operation button 46a, the state of the operation button 46a is the first state.

The control unit 40 determines a state of the digital value DI2 corresponding to the analog voltage AN2 based on the state of the operation button 46a. For example, when the operation button 46a is pressed, the control unit 40 determines that the digital value DI2 is valid. When the operation button 46a is not pressed, the control unit 40 determines that the digital value DI2 is invalid.

When the control unit 40 determines that the digital value DI2 is valid, the control unit 40 calculates the bending control value CO based on only the digital value DI2. Alternatively, the control unit 40 calculates the bending control value CO based on the digital value DI1 and the digital value DI2. When the control unit 40 determines that the digital value DI2 is invalid, the control unit 40 calculates the bending control value CO based on only the digital value DI1.

When the endoscope device 10 is powered on, the state of the operation button 46a may be set to the second state. At this time, the control unit 40 may determine that the digital value DI2 is invalid. When the state of the operation button 46a is the second state and the operation button 46a is pressed, the state of the operation button 46a may change from the second state to the first state. At this time, the control unit 40 may determine that the digital value DI2 is valid. Until the operation button 46a is pressed again, the state of the operation button 46a may be maintained to be the first state.

When the operation button 46a is pressed again, the state of the operation button 46a may change from the first state to the second state. At this time, the control unit 40 may determine that the digital value DI2 is invalid. Until the operation button 46a is pressed again, the state of the operation button 46a may be maintained to be the second state. As described above, the state of the operation button 46a may be switched between the first state and the second state each time the operation button 46a is pressed.

The operation device 4a may include a slide switch or a rotary switch instead of the operation button 46a. For example, the first state is a state in which the slide switch or the rotary switch is located at a first position. For example, the second state is a state in which the slide switch or the rotary switch is located at a second position different from the first position.

Each aspect of the present invention may include the following modified example. The operation button 46a (switch) is disposed on the housing 48. The state of the operation button 46a is switched between a first state and a second state. When the state of the operation button 46a is the first state, the digital value DI2 in accordance with the analog voltage AN2 (second signal) is valid. When the state of the operation button 46a is the second state, the digital value DI2 is invalid. The control unit 40 calculates a bending control value (second control value) only when the digital value DI2 is valid.

There is a possibility that a user unintentionally tilts the operation device 4a. In a case in which the digital value DI2 is always valid, the insertion unit 2 may be bent against the user's intention. In the second embodiment, the user can intentionally set the state of the operation button 46a to the first state. The control unit 40 calculates a bending control value by using the digital value DI2 only when the digital value DI2 is valid. Therefore, it is possible to prevent the insertion unit 2 from bending in accordance with the movement of the operation device 4a which unintended by the user.

### Third Embodiment

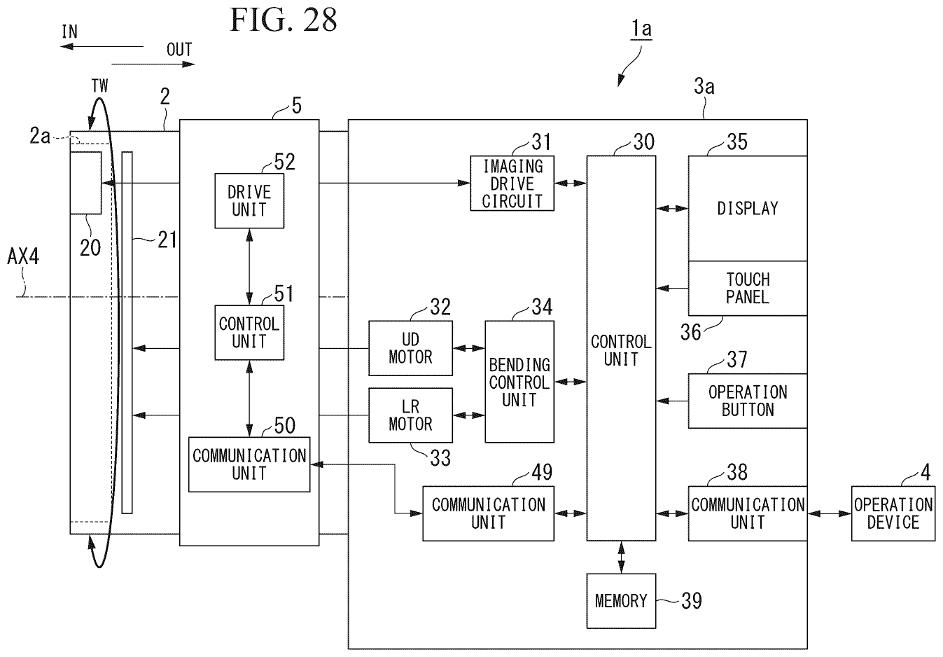

A third embodiment of the present invention will be described. FIG. 28 shows a configuration of an endoscope system 1a according to the third embodiment. The same configuration as that shown in FIG. 1 will not be described. The endoscope system 1a shown in FIG. 28 includes an insertion unit 2, a main body unit 3a, an operation device 4, and an insertion device 5 (automatic insertion machine). The insertion unit 2 is the same as that shown in FIG. 1. The operation device 4 is the same as that shown in FIG. 1.

The main body unit 3a includes a control unit 30, an imaging drive circuit 31, a UD motor 32, an LR motor 33, a bending control unit 34, a display 35, a touch panel 36, an operation button 37, a communication unit 38, a memory 39, and a communication unit 49. The configuration of the main body unit 3a other than the communication unit 49 is the same as that of the main body unit 3 shown in FIG. 1. The communication unit 49 executes wired communication or wireless communication with a communication unit 50 included in the insertion device 5.

For example, the insertion device 5 is fixed to an inspection port (access port). The inspection port is a hole formed in a subject in order to insert the distal end portion 2a of the insertion unit 2 into the subject.

The insertion device 5 includes a communication unit 50, a control unit 51, and a drive unit 52. The communication unit 50 executes wired communication or wireless communication with the communication unit 49 of the main body unit 3a. The control unit 51 controls the communication unit 50 and the drive unit 52.

For example, the drive unit 52 includes a motor and a roller. A rotation axis of the motor is connected to the roller. The roller is disposed at a position at which the roller touches the insertion unit 2. The drive unit 52 rotates the roller by rotating the motor. When the drive unit 52 rotates the roller, the insertion unit 2 moves in a direction IN or a direction OUT parallel to a center axis AX4 of the insertion unit 2 due to the friction force between the roller and the insertion unit 2. When the insertion unit 2 moves in the direction IN, the distal end portion 2a is inserted into a subject. When the insertion unit 2 moves in the direction OUT, the distal end portion 2a is pulled out of the subject.

Alternatively, when the drive unit 52 rotates the roller, the insertion unit 2 rotates in a direction TW around the center axis AX4 of the insertion unit 2. By doing this, the insertion device 5 twists the insertion unit 2.

The control unit 40 calculates an insertion control value based on the digital value DI1 or the digital value DI2. The insertion control value indicates a movement direction and a movement amount of the insertion unit 2. The movement direction indicates the direction IN, the direction OUT, or the direction TW. The control unit 40 outputs the insertion control value to the communication unit 49. The communication unit 49 transmits the insertion control value to the communication unit 50. The communication unit 50 outputs the insertion control value to the control unit 51. The control unit 51 generates a drive signal which is used for driving the drive unit 52, based on the insertion control value. The control unit 51 outputs the drive signal to the drive unit 52 and drives the drive unit 52. The drive unit 52 rotates the motor based on the drive signal.

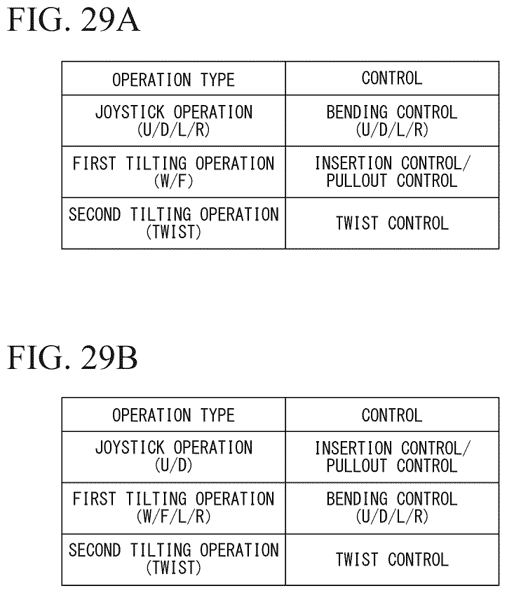

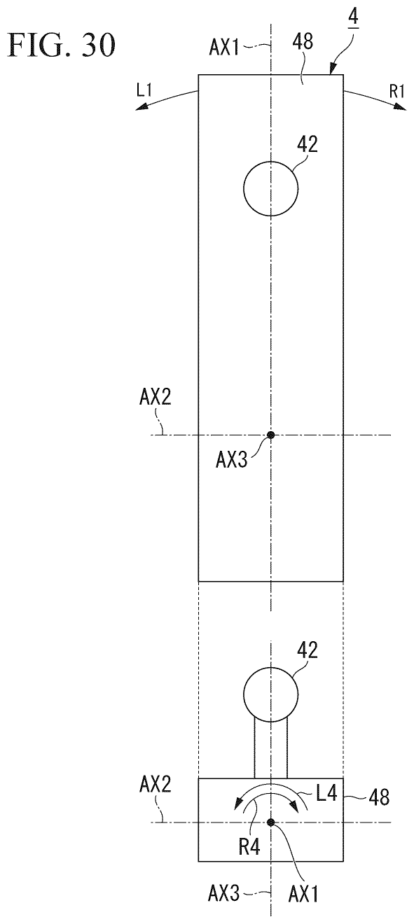

Control in the third embodiment will be described by using FIG. 29A, FIG. 29B, and FIG. 30. FIG. 29A FIG. and 29B show examples of a relationship between operations performed by a user and details of the control.

FIG. 29A shows a first example of the control in accordance with a combination of the joystick operation and the tilting operation. The joystick operation is associated with the bending control (first control). A user performs the joystick operation by tilting the joystick 42 in the upward direction U2, the downward direction D2, the left direction L2, or the right direction R2 shown in FIG. 4. When the user performs the joystick operation, the control unit 40 calculates a bending control value by using the method in the first embodiment. The insertion unit 2 is bent upward, downward, leftward, or right ward.

Two types of tilting operations, in other words, a first tilting operation and a second tilting operation are prepared. The first tilting operation is associated with insertion control and pullout control (second control). The insertion control is executed in order to move the insertion unit 2 in the direction IN and insert the distal end portion 2a into a subject. The pullout control is executed in order to move the insertion unit 2 in the direction OUT and pull the distal end portion 2a out of the subject. The user performs the first tilting operation by tilting the operation device 4 in the front direction F1 or the rear direction W1 shown in FIG. 3.

FIG. 30 is a front view and a side view of the operation device 4. An axis AX1, an axis AX2, and an axis AX3 are shown in FIG. 30. Each axis is the same as that shown in FIG. 2.

When a user performs the first tilting operation, the motion sensor 44 outputs an analog voltage AN2 (second signal) in accordance with a rotation direction and a rotation amount of the housing 48 around the axis AX2. The ADC 45 converts the analog voltage AN2 into a digital value DI2. The control unit 40 calculates an insertion control value based on the digital value DI2. The insertion device 5 moves the insertion unit 2 in the direction IN or the direction OUT.

For example, when the user tilts the operation device 4 in the rear direction W1 shown in FIG. 3, the control unit 40 calculates an insertion control value used for moving the insertion unit 2 in the direction IN. The insertion device 5 moves the insertion unit 2 in the direction IN. For example, when the user tilts the operation device 4 in the front direction F1 shown in FIG. 3, the control unit 40 calculates an insertion control value used for moving the insertion unit 2 in the direction OUT. The insertion device 5 moves the insertion unit 2 in the direction OUT.

The second tilting operation is associated with twist control (third control). The twist control is executed in order to twist the insertion unit 2 in the direction TW. A user performs the second tilting operation by tilting the operation device 4 around the axis AX1 parallel to the longitudinal direction of the housing 48.

When the user performs the second tilting operation, the motion sensor 44 outputs an analog voltage AN2 (third signal) in accordance with a rotation direction and a rotation amount of the housing 48 around the axis AX1. The ADC 45 converts the analog voltage AN2 into a digital value DI2. The control unit 40 calculates an insertion control value based on the digital value DI2. The insertion device 5 twists the insertion unit 2 in the direction TW.

For example, when the user tilts the operation device 4 in a right direction R4 shown in FIG. 30, the control unit 40 calculates an insertion control value used for twisting the insertion unit 2 rightward. The insertion device 5 twists the insertion unit 2 rightward. For example, when the user tilts the operation device 4 in a left direction L4 shown in FIG. 30, the control unit 40 calculates an insertion control value used for twisting the insertion unit 2 leftward. The insertion device 5 twists the insertion unit 2 leftward.

When the user tilts the operation device 4 in a left direction L1 or a right direction R1 shown in FIG. 30, the control unit 40 does not calculate a control value in accordance with a rotation amount of the housing 48 around the axis AX3.

FIG. 29B shows a second example of the control in accordance with a combination of the joystick operation and the tilting operation. The joystick operation is associated with the insertion control and the pullout control (first control). A user performs the joystick operation by tilting the joystick 42 in the upward direction U2, the downward direction D2, the left direction L2, or the right direction R2 shown in FIG. 4. When the user performs the joystick operation, the control unit 40 calculates an insertion control value based on both a direction in which the user tilts the joystick 42 and an angle of the joystick 42.

For example, when the user tilts the joystick 42 in the upward direction U2 shown in FIG. 4, the control unit 40 calculates an insertion control value used for moving the insertion unit 2 in the direction IN. The insertion device 5 moves the insertion unit 2 in the direction IN. For example, when the user tilts the joystick 42 in the downward direction D2 shown in FIG. 4, the control unit 40 calculates an insertion control value used for moving the insertion unit 2 in the direction OUT. The insertion device 5 moves the insertion unit 2 in the direction OUT.

The first tilting operation is associated with the bending control (second control). The user performs the first tilting operation by tilting the operation device 4 in the left direction L1 or the right direction R1 shown in FIG. 2. Alternatively, the user performs the first tilting operation by tilting the operation device 4 in the front direction F1 or the rear direction W1 shown in FIG. 3. When the user performs the first tilting operation, the control unit 40 calculates a bending control value by using the method in the first embodiment. The insertion unit 2 is bent upward, downward, leftward, or rightward.

The second tilting operation is associated with the twist control (third control). For example, when the user tilts the operation device 4 in the right direction R4, the control unit 40 calculates an insertion control value used for twisting the insertion unit 2 rightward. The insertion device 5 twists the insertion unit 2 rightward. For example, when the user tilts the operation device 4 in the left direction L4, the control unit 40 calculates an insertion control value used for twisting the insertion unit 2 leftward. The insertion device 5 twists the insertion unit 2 leftward.

When the user tilts the joystick 42 in the left direction L2 or the right direction R2 shown in FIG. 4, the control unit 40 does not calculate a control value in accordance with the angle of the joystick 42.

The operation device 4 may be changed to the operation device 4a shown in FIG. 27. The control unit 40 may determine that the digital value DI2 is valid or invalid based on the state of the operation button 46a of the operation device 4a.

Each aspect of the present invention may include the following modified example. The insertion device 5 inserts the distal end portion 2a into an object and pulls the distal end portion 2a out of the object. One of the first control and the second control is executed to bend the insertion unit 2. The other of the first control and the second control is executed to control insertion and pullout of the distal end portion 2a.

Each aspect of the present invention may include the following modified example. The operation device 4 has an elongated shape. The motion sensor 44 generates the analog voltage AN2 (third signal) in accordance with the rotation of the operation device 4 around the axis AX1 parallel to the longitudinal direction of the operation device 4. The insertion device 5 rotates the insertion unit 2 around the center axis AX4 of the insertion unit 2. The control unit 40 calculates, based on the digital value DI2 corresponding to the analog voltage AN2, a third control value used to control rotation of the insertion unit 2.

In the third embodiment, a user can insert the distal end portion 2a into a subject or can pull the distal end portion 2a out of the subject by performing the joystick operation or the tilting operation. In addition, the user can twist the insertion unit 2 by tilting the operation device 4.

### Fourth Embodiment

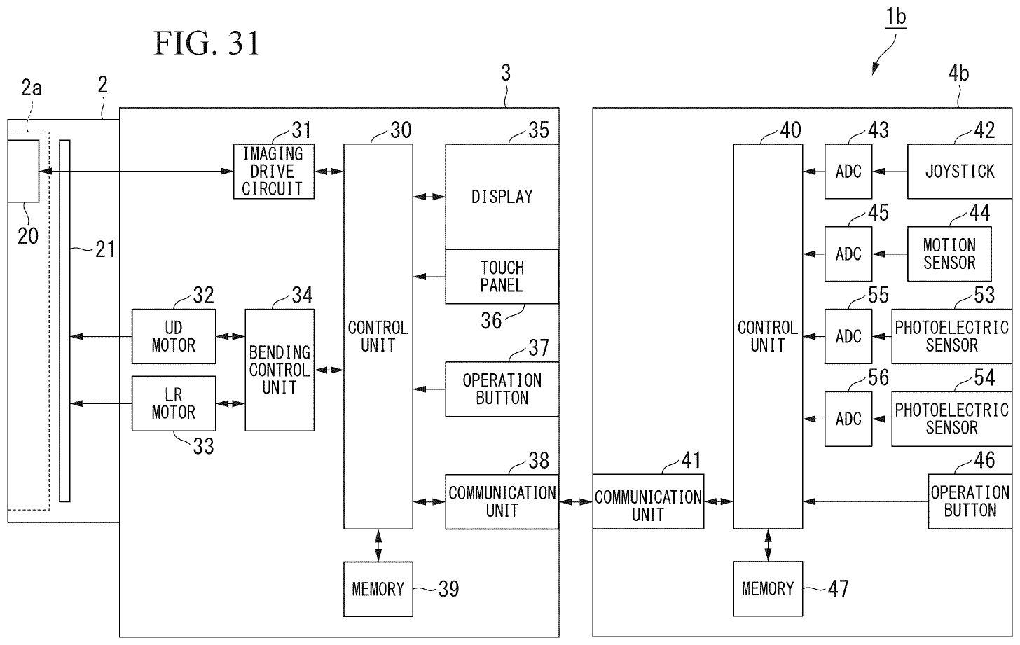

A fourth embodiment of the present invention will be described. FIG. 31 shows a configuration of an endoscope system 1b according to the fourth embodiment. The same configuration as that shown in FIG. 1 will not be described. The endoscope system 1b shown in FIG. 31 includes an insertion unit 2, a main body unit 3, and an operation device 4b. The insertion unit 2 is the same as that shown in FIG. 1. The main body unit 3 is the same as that shown in FIG. 1.

The operation device 4b includes a control unit 40, a communication unit 41, a joystick 42, an ADC 43, a motion sensor 44, an ADC 45, an operation button 46, a memory 47, a photoelectric sensor 53, a photoelectric sensor 54, an ADC 55, and an ADC 56. The configuration of the operation device 4b excluding the photoelectric sensor 53, the photoelectric sensor 54, the ADC 55, and the ADC 56 is the same as that of the operation device 4 shown in FIG. 1.

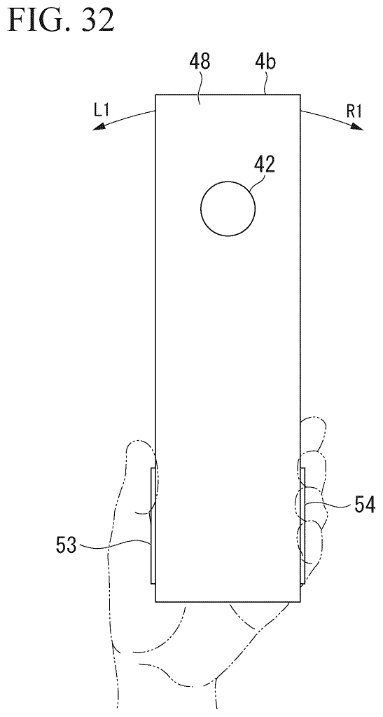

FIG. 32 shows an external appearance of the operation device 4b. The operation device 4b includes a housing 48. The photoelectric sensor 53 and the photoelectric sensor 54 are disposed on the housing 48.

In the example shown in FIG. 32, the photoelectric sensor 53 is disposed on the left side surface of the housing 48, and the photoelectric sensor 54 is disposed on the right side surface of the housing 48. A user holds the operation device 4b such that the palm thereof touches the rear surface of the operation device 4b. The photoelectric sensor 53 and the photoelectric sensor 54 are disposed at positions at which the photoelectric sensor 53 and the photoelectric sensor 54 are covered by the user's palm or fingers when he/she holds the operation device 4b. The positions of the photoelectric sensor 53 and the photoelectric sensor 54 are not limited to those shown in FIG. 32. The operation device 4b may include two or more photoelectric sensors 53. The operation device 4b may include two or more photoelectric sensors 54.

The photoelectric sensor 53 includes a light-emitting device and a light-receiving device. The light-emitting device irradiates an object with light. The light-receiving device receives light reflected by the object. When the user is holding the operation device 4b, the object is the user's palm or finger. The photoelectric sensor 53 outputs a first analog signal to the ADC 55 in accordance with the area covered by the user's palm or finger. The photoelectric sensor 54 is constituted similarly to the photoelectric sensor 53. The photoelectric sensor 54 outputs a second analog signal o the ADC 56 in accordance with the area covered by the user's palm or finger. Touch sensors or tactile sensors may be used instead of the photoelectric sensor 53 and the photoelectric sensor 54.

The ADC 55 converts the first analog signal output from the photoelectric sensor 53 into a first digital value and outputs the first digital value to the control unit 40. The ADC 56 converts the second analog signal output from the photoelectric sensor 54 into a second digital value and outputs the second digital value into the control unit 40.

The photoelectric sensor 53 may include the ADC 55 and output the first digital value. The photoelectric sensor 54 may include the ADC 56 and output the second digital value.

The control unit 40 determines whether a user is holding the operation device 4b with the left hand or holds the operation device 4b with the right hand based on the first digital value and the second digital value. For example, when the user is holding the operation device 4b with the left hand, almost the entire photoelectric sensor 53 is covered by his/her palm and part of the photoelectric sensor 54 is covered by the his/her two or more fingers. On the other hand, when the user is holding the operation device 4b with the right hand, part of the photoelectric sensor 53 is covered by his/her two or more fingers and almost the entire photoelectric sensor 54 is covered by his/her palm. The control unit 40 can determine the type of hand with which the user is holding the operation device 4b based on the first digital value and the second digital value.

The operation device 4b may include either the photoelectric sensor 53 or the photoelectric sensor 54. In a case in which the operation device 4b does not include the photoelectric sensor 54, the operation device 4b may include two or more photoelectric sensors 53. In a case in which the operation device 4b does not include the photoelectric sensor 53, the operation device 4b may include two or more photoelectric sensors 54. For example, in a case in which the operation device 4b does not include the photoelectric sensor 54, the control unit 40 may determine whether the user is holding the operation device 4b with the left hand or holds the operation device 4b with the right hand based on the first digital value.

When the user is holding the operation device 4b with the left hand, he/she feels it easier to tilt the operation device 4b in a right direction R1 shown in FIG. 32 than in a left direction L1 shown in FIG. 32. On the other hand, when the user is holding the operation device 4b with the right hand, he/she feels it easier to tilt the operation device 4b in the left direction L1 than in the right direction R1. When the user performs the tilting operation, the control unit 40 corrects a bending control value in accordance with the type of hand with which the user is holding the operation device 4b.

The memory 47 stores first bending information and second bending information indicating a relationship between a rotation amount of the housing 48 and a bending control value. When the user is holding the operation device 4b with the left hand, the control unit 40 uses the first bending information to calculate a bending control value in accordance with the tilting operation. When the user is holding the operation device 4b with the right hand, the control unit 40 uses the second bending information to calculate a bending control value in accordance with the tilting operation.

The memory 47 may store the first bending information and the second bending information in a process in which the endoscope system 1b is manufactured. The endoscope system 1b may connect to an external server storing the first bending information and the second bending information and may download the first bending information and the second bending information from the external server. The memory 47 may store the first bending information and the second bending information downloaded from the external server.

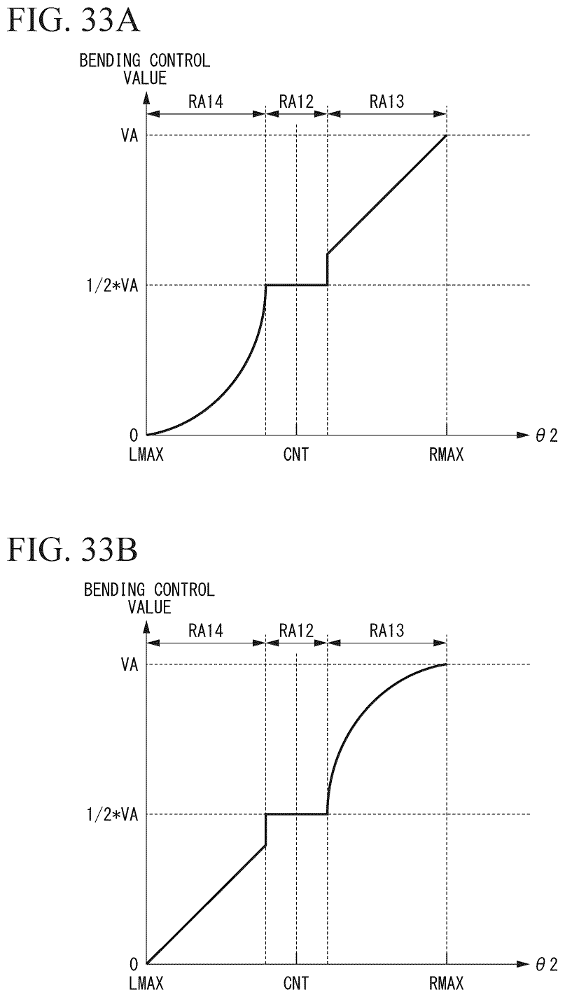

FIG. 33A is a graph showing a relationship between a tilted state of the operation device 4b and a bending control value. The graph shown in FIG. 33A is an example of the first bending information. The horizontal axis in the graph shown in FIG. 33A indicates an angle (θ2) of the operation device 4b, and the vertical axis in the graph shown in FIG. 33A indicates a bending control value. The angle of the operation device 4b is equivalent to a rotation amount of the housing 48.

The angle of the operation device 4b ranges from CNT to LMAX or ranges from CNT to RMAX. When the operation device 4b is tilted by 90 degrees or more in the left direction L3 shown in FIG. 17, the angle of the operation device 4b is LMAX. When the operation device 4b is tilted by 90 degrees or more in the right direction R3 shown in FIG. 17, the angle of the operation device 4b is RMAX. When a user is not tilting the operation device 4b, the angle of the operation device 4b is CNT.

The angle of the operation device 4b is included in either a range RA12, a range RA13, or a range RA14. When the angle of the operation device 4b is included in the range RA12, the bending control value is a constant value (½*VA). Even when the angle of the operation device 4b changes slightly in the vicinity of CNT, the bending control value is a constant value. Therefore, it is possible to prevent a bending amount of the insertion unit 2 from changing in accordance with the movement of the operation device 4b, which is unintended by the user.

Characteristics of the bending control value in accordance with the angle of the operation device 4b in the range RA14 are different from those of the bending control value in accordance with the angle of the operation device 4b in the range RA13. When the angle of the operation device 4b is included in the range RA13 on the right side of the range RA12, the bending control value linearly changes in accordance with the angle of the operation device 4b. When the angle of the operation device 4b is included in the range RA14 on the left side of the range RA12, the bending control value curvilinearly changes in accordance with the angle of the operation device 4b. When the angle of the operation device 4b in the range RA14 changes in the vicinity of the range RA12, the bending control value greatly changes. When the angle of the operation device 4b in the range RA14 changes in the vicinity of LMAX, the bending control value slightly changes.

FIG. 33B is a graph showing a relationship between a tilted state of the operation device 4b and a bending control value. The graph shown in FIG. 33B is an example of the second bending information. The horizontal axis in the graph shown in FIG. 33B indicates an angle (θ2) of the operation device 4b, and the vertical axis in the graph shown in FIG. 33B indicates a bending control value.

The angle of the operation device 4b is LMAX or more and is RMAX or less. When a user is not tilting the operation device 4b, the angle of the operation device 4b is CNT. The angle of the operation device 4b is included in either a range RA12, a range RA13, or a range RA14. When the angle of the operation device 4b is included in the range RA12, the bending control value is a constant value (½*VA).

When the angle of the operation device 4b is included in the range RA14 on the left side of the range RA12, the bending control value linearly changes in accordance with the angle of the operation device 4b. When the angle of the operation device 4b is included in the range RA13 on the right side of the range RA12, the bending control value curvilinearly changes in accordance with the angle of the operation device 4b. When the angle of the operation device 4b in the range RA13 changes in the vicinity of the range RA12, the bending control value greatly changes. When the angle of the operation device 4b in the range RA13 changes in the vicinity of RMAX, the bending control value slightly changes.

When a user tilts the operation device 4b in the left direction L1 or the right direction R1 shown in FIG. 2, the control unit 40 calculates a bending control value by using the first bending information or the second bending information. When the user tilts the operation device 4b in the upward direction U1 or the downward direction D1 shown in FIG. 2, the control unit 40 can calculate a bending control value by using similar information to the first bending information or the second bending information. When the user tilts the operation device 4b in the front direction F1 or the rear direction W1 shown in FIG. 3, the control unit 40 can calculate a bending control value by using similar information to the first bending information or the second bending information.

The operation device 4b may include the operation button 46a shown in FIG. 27. The control unit 40 may determine that the digital value DI2 is valid or invalid based on the state of the operation button 46a.

In the endoscope system 1a shown in FIG. 28, the operation device 4 may be changed to the operation device 4b. When a user performs the first tilting operation, the control unit 40 may calculate a bending control value or an insertion control value by using similar information to the first bending information or the second bending information. When the user performs the second tilting operation, the control unit 40 may calculate an insertion control value by using similar information to the first bending information or the second bending information.

Each aspect of the present invention may include the following modified example. The operation device 4b includes the photoelectric sensor 53 (photoelectric sensor 54) that outputs a signal in accordance with whether a user is holding the operation device 4b with the left hand or is holding the operation device 4b with the right hand. The control unit 40 determines whether the user is holding the operation device 4b with the left hand or is holding the operation device 4b with the right hand based on a digital value corresponding to the signal output from the photoelectric sensor 53. When it is determined that the user is holding the operation device 4b with the left hand, the control unit 40 calculates a bending control value by using first bending information (first information) indicating a relationship between the rotation amount of the operation device 4b and the bending control value (second control value). When it is determined that the user is holding the operation device 4b with the right hand, the control unit 40 calculates a bending control value by using second bending information (second information) indicating a relationship between a rotation amount of the operation device 4b and the bending control value (second control value). The second bending information is different from the first bending information.

In the fourth embodiment, the control unit 40 corrects a bending control value based on the type of hand with which a user is holding the operation device 4b. The endoscope system 1b can reduce a user's burden in the tilting operation.

### Fifth Embodiment

A fifth embodiment of the present invention will be described. In the fifth embodiment, the endoscope system 1 shown in FIG. 1 is used.

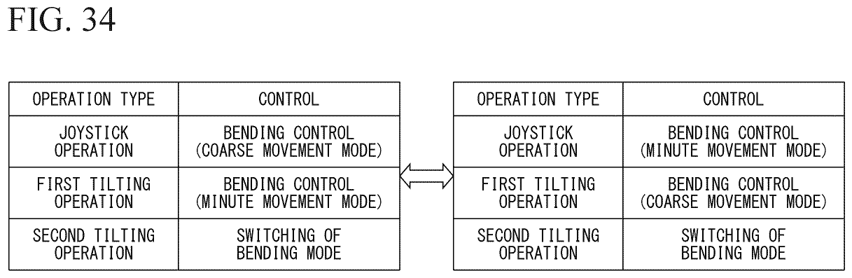

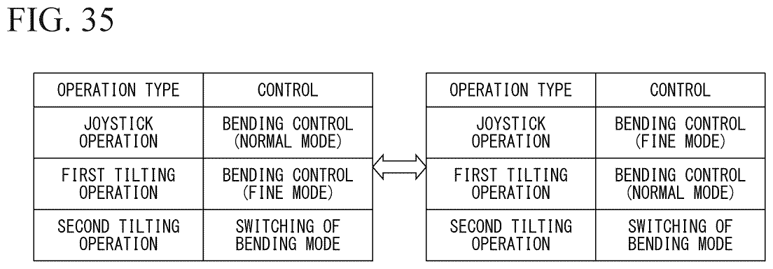

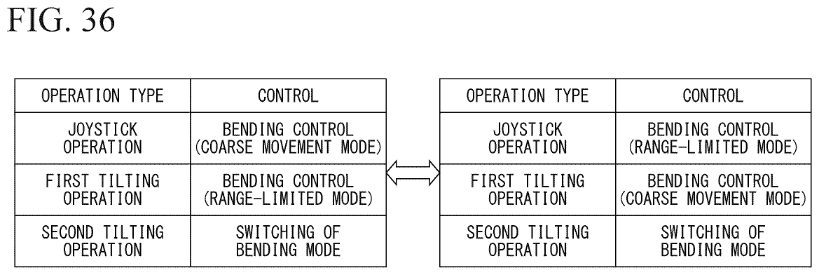

Control in the fifth embodiment will be described by using FIG. 34, FIG. 35, and FIG. 36. FIG. 34, FIG. 35, and FIG. 36 show examples of a relationship between operations performed by a user and details of the control.

FIG. 34 shows a first example of the control in accordance with a combination of the joystick operation and the tilting operation. The joystick operation is associated with the bending control (first control). A user performs the joystick operation by tilting the joystick 42 in the upward direction U2, the downward direction D2, the left direction L2, or the right direction R2 shown in FIG. 4. When the user performs the joystick operation, the control unit 40 calculates a bending control value by using the method in the first embodiment. The insertion unit 2 is bent upward, downward, leftward, or rightward.

Two types of tilting operations, in other words, a first tilting operation and a second tilting operation are prepared. The first tilting operation is associated with the bending control (second control). The user performs the first tilting operation by tilting the operation device 4 in the left direction L1 or the right direction R1 shown in FIG. 2. Alternatively, the user performs the first tilting operation by tilting the operation device 4 in the front direction F1 or the rear direction W1 shown in FIG. 3. When the user performs the first tilting operation, the control unit 40 calculates the bending control value by using the method in the first embodiment. The insertion unit 2 is bent upward, downward, leftward, or rightward.

The second tilting operation is associated with control (third control) of switching bending modes. The user performs the second tilting operation by tilting the operation device 4 in the left direction L4 or the right direction R4 shown in FIG. 30.

When the user performs the second tilting operation, the motion sensor 44 outputs an analog voltage AN2 (third signal) in accordance with a rotation direction and a rotation amount of the housing 48 around the axis AX1 shown in FIG. 30. The ADC 45 converts the analog voltage AN2 into a digital value DI2. The control unit 40 determines whether to switch the bending modes based on the digital value DI2. For example, when the rotation amount of the housing 48 indicated by the digital value DI2 is greater than a predetermined amount, the control unit 40 determines to switch the bending modes. For example, when the rotation amount of the housing 48 indicated by the digital value DI2 is less than or equal to the predetermined amount, the control unit determines not to switch the bending modes.

For example, the joystick operation is associated with the bending control (first control) in the coarse movement mode. The first tilting operation is associated with the bending control (second control) in the minute movement mode. When the user performs the joystick operation, the insertion unit 2 is bent by a bending amount in the coarse movement mode. When the user performs the first tilting operation, the insertion unit 2 is bent by a bending amount in the minute movement mode.

When the control unit 40 determines to switch the bending modes, the control unit 40 changes allocation of the bending modes for the first control and the second control. Specifically, the control unit 40 allocates the bending control in the minute movement mode to the first control and allocates the bending control in the coarse movement mode to the second control. When the user performs the joystick operation, the insertion unit 2 is bent by a bending amount in the minute movement mode. When the user performs the first tilting operation, the insertion unit 2 is bent by a bending amount in the coarse movement mode.

FIG. 35 shows a second example of the control in accordance with a combination of the joystick operation and the tilting operation. The joystick operation is associated with the bending control (first control) similarly to the first example. The first tilting operation is associated with the bending control (second control) similarly to the first example. The second tilting operation is associated with the control (third control) of switching the bending modes similarly to the first example.

For example, the joystick operation is associated with the bending control (first control) in the normal mode. The first tilting operation is associated with the bending control (second control) in the fine mode. When a user performs the joystick operation, the insertion unit 2 is bent by a bending amount in the normal mode. When the user performs the first tilting operation, the insertion unit 2 is bent by a bending amount in the fine mode.

When the control unit 40 determines to switch the bending modes, the control unit 40 changes allocation of the bending modes for the first control and the second control. Specifically, the control unit 40 allocates the bending control in the fine mode to the first control and allocates the bending control in the normal mode to the second control. When the user performs the joystick operation, the insertion unit 2 is bent by a bending amount in the fine mode. When the user performs the first tilting operation, the insertion unit 2 is bent by a bending amount in the normal mode.

FIG. 36 shows a third example of the control in accordance with a combination of the joystick operation and the tilting operation. The joystick operation is associated with the bending control (first control) similarly to the first example. The first tilting operation is associated with the bending control (second control) similarly to the first example. The second tilting operation is associated with the control (third control) of switching the bending modes similarly to the first example.

For example, the joystick operation is associated with the bending control (first control) in the coarse movement mode. The first tilting operation is associated with the bending control (second control) in the range-limited mode. When a user performs the joystick operation, the insertion unit 2 is bent by a bending amount in the coarse movement mode. When the user performs the first tilting operation, the insertion unit 2 is bent by a bending amount in the range-limited mode.

When the control unit 40 determines to switch the bending modes, the control unit 40 changes allocation of the bending modes for the first control and the second control. Specifically, the control unit 40 allocates the bending control in the range-limited mode to the first control and allocates the bending control in the coarse movement mode to the second control. When the user performs the joystick operation, the insertion unit 2 is bent by a bending amount in the range-limited mode. When the user performs the first tilting operation, the insertion unit 2 is bent by a bending amount in the coarse movement mode.

The operation device 4 may be changed to the operation device 4a shown in FIG. 27. The control unit 40 may determine that the digital value DI2 is valid or invalid based on the state of the operation button 46a of the operation device 4a.

The endoscope system 1 may include the insertion device 5 shown in FIG. 28. The joystick operation or the first tilting operation may be associated with the insertion control and the pullout control.

The operation device 4 may be changed to the operation device 4b shown in FIG. 31. When a user performs the joystick operation or the first tilting operation, the control unit 40 may calculate a bending control value by using similar information to the first bending information or the second bending information in the fourth embodiment.

Each aspect of the present invention may include the following modified example. The motion sensor 44 generates the analog voltage AN2 (third signal) in accordance with the rotation of the operation device 4. The control unit 40 allocates one of control of a first function and control of a second function to the first control based on the digital value DI2 corresponding to the analog voltage AN2. The control unit 40 allocates the other of control of the first function and control of the second function to the second control based on the digital value DI2.

In the fifth embodiment, the control unit 40 changes allocation of the bending modes for the first control and the second control. A user can easily change details of the first control and the second control.

### Sixth Embodiment

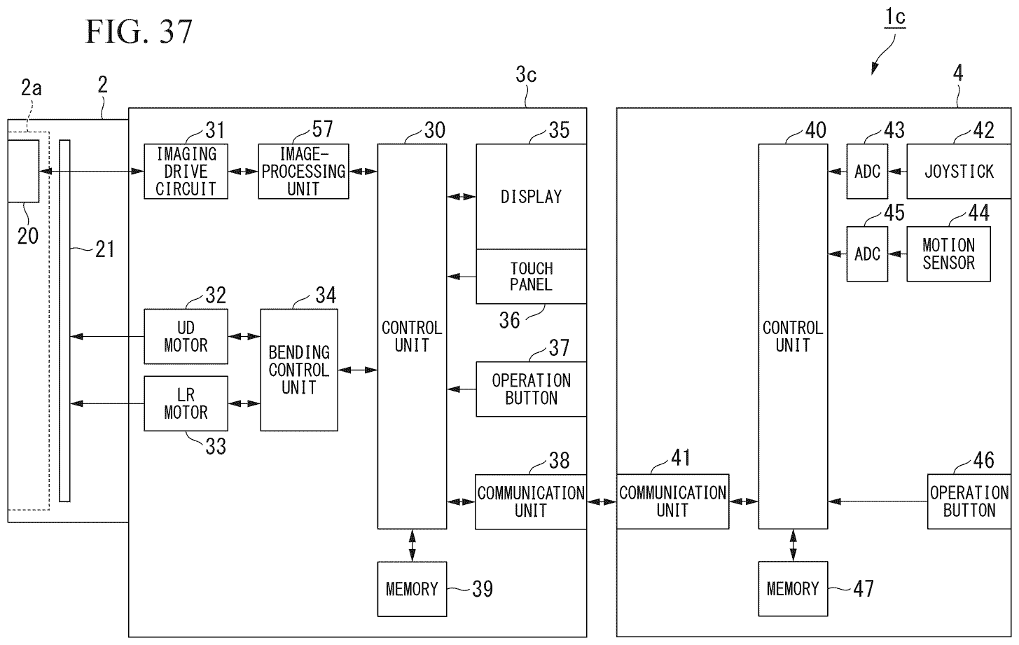

A sixth embodiment of the present invention will be described. FIG. 37 shows a configuration of an endoscope system 1c according to the sixth embodiment. The same configuration as that shown in FIG. 1 will not be described. The endoscope system 1c shown in FIG. 37 includes an insertion unit 2, a main body unit 3c, and an operation device 4. The insertion unit 2 is the same as that shown in FIG. 1. The operation device 4 is the same as that shown in FIG. 1.

The main body unit 3c includes a control unit 30, an imaging drive circuit 31, a UD motor 32, an LR motor 33, a bending control unit 34, a display 35, a touch panel 36, an operation button 37, a communication unit 38, a memory 39, and an image-processing unit 57. The configuration of the main body unit 3c other than the image-processing unit 57 is the same as that of the main body unit 3 shown in FIG. 1.

The imaging drive circuit 31 outputs an image, which is output from the imaging unit 20 of the insertion unit 2, to the image-processing unit 57. The image-processing unit 57 performs image processing on the image output from the imaging drive circuit 31 and changes the state of the image. The image processing is brightness adjustment, zoom processing, noise reduction, color density adjustment, contour enhancement, and the like. The image-processing unit 57 may be constituted by at least one of a processor and a logic circuit. The image-processing unit 57 may include one or more processors. The image-processing unit 57 may include one or more logic circuits.

The image-processing unit 57 outputs the image on which the image processing has been performed to the control unit 30. The control unit 30 outputs the image to the display 35.

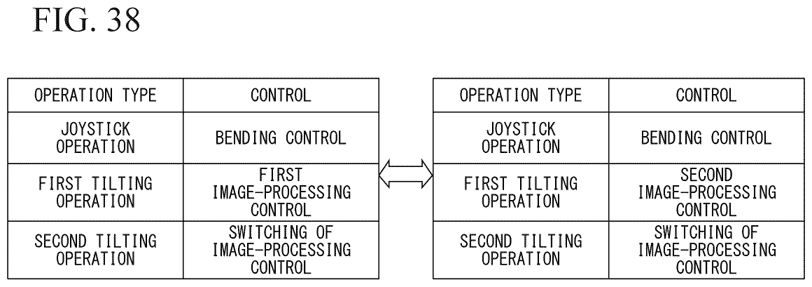

Control in the sixth embodiment will be described by using FIG. 38. FIG. 38 shows an example of a relationship between operations performed by a user and details of the control.

The joystick operation is associated with the bending control (first control). A user performs the joystick operation by tilting the joystick 42 in the upward direction U2, the downward direction D2, the left direction L2, or the right direction R2 shown in FIG. 4. When the user performs the joystick operation, the control unit 40 calculates a bending control value by using the method in the first embodiment. The insertion unit 2 is bent upward, downward, leftward, or rightward.

Two types of tilting operations, in other words, a first tilting operation and a second tilting operation are prepared. The first tilting operation is associated with first image-processing control (second control). The first image-processing control is executed in order to control the image processing performed by the image-processing unit 57.

The user performs the first tilting operation by tilting the operation device 4 in the left direction L1 or the right direction R1 shown in FIG. 2. Alternatively, the user performs the first tilting operation by tilting the operation device 4 in the front direction F1 or the rear direction W1 shown in FIG. 3. When the user performs the first tilting operation, the control unit 40 calculates an image-processing control value in accordance with the first tilting operation. The image-processing control value indicates a parameter of the image processing performed by the image-processing unit 57. For example, the control unit 40 calculates the image-processing control value by using a similar method to that of calculating a bending control value.

The control unit 40 outputs the image-processing control value to the communication unit 41. The communication unit 41 transmits the image-processing control value to the communication unit 38. The communication unit 38 outputs the image-processing control value to the control unit 30. The control unit 30 controls the image-processing unit 57 based on the image-processing control value.

The second tilting operation is associated with control (third control) for switching types of image-processing control. The user performs the second tilting operation by tilting the operation device 4 in the left direction L4 or the right direction R4 shown in FIG. 30.

When the user performs the second tilting operation, the motion sensor 44 outputs an analog voltage AN2 (third signal) in accordance with a rotation direction and a rotation amount of the housing 48 around the axis AX1 shown in FIG. 30. The ADC 45 converts the analog voltage AN2 into a digital value DI2. The control unit 40 determines whether to switch the type of image-processing control based on the digital value DI2. For example, when the rotation amount of the housing 48 indicated by the digital value DI2 is greater than a predetermined amount, the control unit 40 determines to switch the types of image-processing control. For example, when the rotation amount of the housing 48 indicated by the digital value DI2 is less than or equal to the predetermined amount, the control unit 40 determines not to switch the types of image-processing control.

For example, the first image-processing control is control of the brightness adjustment. When the user performs the first tilting operation, the control unit 40 calculates an image-processing control value of the brightness adjustment. The control unit 30 updates a parameter of the brightness adjustment performed by the image-processing unit 57 with the image-processing control value.

When the control unit 40 determines to switch the types of image-processing control, the control unit 40 changes allocation of the image-processing control for the second control. Specifically, the control unit 40 allocates second image-processing control to the second control. For example, the second image-processing control is control of the noise reduction. When the user performs the first tilting operation, the control unit 40 calculates an image-processing control value of the noise reduction. The control unit 30 updates a parameter of the noise reduction performed by the image-processing unit 57 with the image-processing control value. Each time the user performs the second tilting operation, the control unit 40 changes the allocation of the image-processing control for the second control.

The operation device 4 may be changed to the operation device 4a shown in FIG. 27. The control unit 40 may determine that the digital value DI2 is valid or invalid based on the state of the operation button 46a of the operation device 4a.

The endoscope system 1c may include the insertion device 5 shown in FIG. 28. The joystick operation may be associated with the insertion control and the pullout control.

The operation device 4 may be changed to the operation device 4b shown in FIG. 31. When a user performs the joystick operation or the first tilting operation, the control unit 40 may calculate a bending control value or an image-processing control value by using similar information to the first bending information or the second bending information in the fourth embodiment.

Each aspect of the present invention may include the following modified example. The imaging unit 20 (image sensor) generates an image based on an optical image acquired by the insertion unit 2. One of the first control and the second control is executed to control image processing of changing the state of the image.

In the sixth embodiment, a user can intuitively change a control value of image processing by tilting the operation device 4.

### Reference Embodiment

A reference embodiment of the present invention will be described. In the reference, the endoscope system 1c shown in FIG. 37 is used.

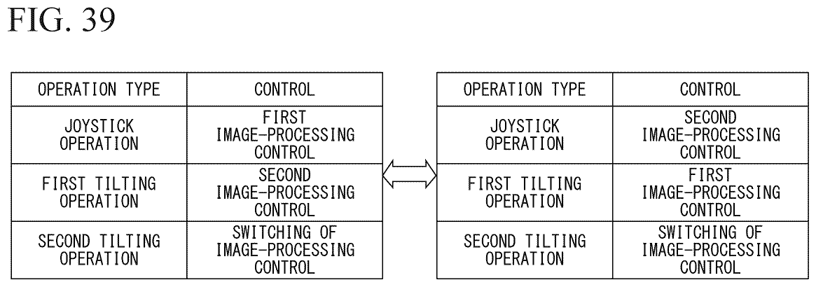

Control in the reference embodiment will be described below by using FIGS. 39 to 42. FIG. 39 shows a first example of a relationship between operations performed by a user and details of the control.

The joystick operation is associated with first image-processing control (first control). The first image-processing control is executed in order to control the image processing performed by the image-processing unit 57. A user performs the joystick operation by tilting the joystick 42 in the upward direction U2, the downward direction D2, the left direction L2, or the right direction R2 shown in FIG. 4. When the user performs the joystick operation, the control unit 40 calculates the first image-processing control value in accordance with the joystick operation. For example, the control unit calculates the first image-processing control value by using a similar method to that of calculating a bending control value. The first image-processing control value indicates a parameter of the image processing performed by the image-processing unit 57.

Two types of tilting operations, in other words, a first tilting operation and a second tilting operation are prepared. The first tilting operation is associated with second image-processing control (second control). The second image-processing control is executed in order to control the image processing performed by the image-processing unit 57.

The user performs the first tilting operation by tilting the operation device 4 in the left direction L1 or the right direction R1 shown in FIG. 2. Alternatively, the user performs the first tilting operation by tilting the operation device 4 in the front direction F1 or the rear direction W1 shown in FIG. 3. When the user performs the first tilting operation, the control unit 40 calculates a second image-processing control value in accordance with the first tilting operation. The second image-processing control value indicates a parameter of the image processing performed by the image-processing unit 57. For example, the control unit 40 calculates the second image-processing control value by using a similar method to that of calculating a bending control value.

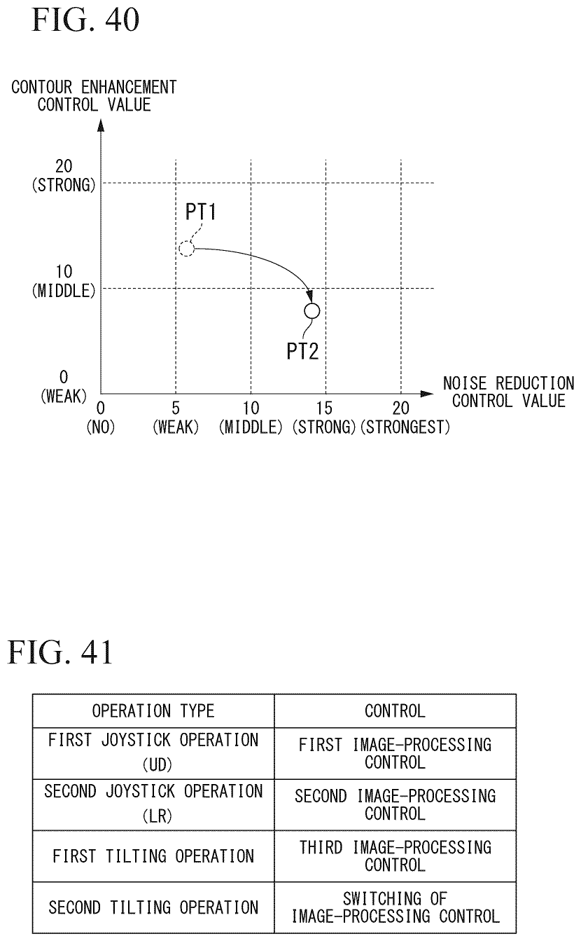

FIG. 40 is a graph showing an example of a first image-processing control value and a second image-processing control value. The horizontal axis in the graph shown in FIG. 40 indicates the first image-processing control value, and the vertical axis in the graph shown in FIG. 40 indicates the second image-processing control value. In the example shown in FIG. 40, the first image-processing control value is a control value of the noise reduction, and the second image-processing control value is a control value of the contour enhancement.

Before a user performs the joystick operation and the first tilting operation, the first image-processing control value and the second image-processing control value are values at a point PT1. The user can simultaneously perform the joystick operation and the first tilting operation. After the user performs the joystick operation and the first tilting operation, the first image-processing control value and the second image-processing control value are values at a point PT2.

The second tilting operation is associated with control (third control) of switching types of image-processing control. The user performs the second tilting operation by tilting the operation device 4 in the left direction L4 or the right direction R4 shown in FIG. 30.

When the user performs the second tilting operation, the motion sensor 44 outputs an analog voltage AN2 (third signal) in accordance with a rotation direction and a rotation amount of the housing 48 around the axis AX1 shown in FIG. 30. The ADC 45 converts the analog voltage AN2 into a digital value DI2. The control unit 40 determines whether to switch the types of image-processing control based on the digital value DI2. For example, when the rotation amount of the housing 48 indicated by the digital value DI2 is greater than a predetermined amount, the control unit 40 determines to switch the types of image-processing control. For example, when the rotation amount of the housing 48 indicated by the digital value DI2 is less than or equal to the predetermined amount, the control unit 40 determines not to switch the types of image-processing control.

When the control unit 40 determines to switch the types of image-processing control, the control unit 40 changes allocation of the image-processing control for the first control and the second control. Specifically, the control unit 40 allocates the second image-processing control to the first control and allocates the first image-processing control to the second control. When the user performs the joystick operation, the control unit 40 calculates a second image-processing control value in accordance with the joystick operation. When the user performs the first tilting operation, the control unit 40 calculates a first image-processing control value in accordance with the first tilting operation.

FIG. 41 shows a second example of a relationship between operations performed by a user and details of the control.

Two types of joystick operations, in other words, a first joystick operation and a second joystick operation are prepared. The first joystick operation is associated with first image-processing control (first control). The first image-processing control is executed in order to control the image processing performed by the image-processing unit 57. A user performs the first joystick operation by tilting the joystick 42 in the upward direction U2 or the downward direction D2 shown in FIG. 4. When the user performs the first joystick operation, the control unit 40 calculates a first image-processing control value in accordance with the first joystick operation. The first image-processing control value indicates a parameter of the image processing performed by the image-processing unit 57. For example, the control unit 40 calculates the first image-processing control value by using a similar method to that of calculating a bending control value.

The second joystick operation is associated with second image-processing control (first control). The second image-processing control is executed in order to control the image processing performed by the image-processing unit 57. A user performs the second joystick operation by tilting the joystick 42 in the left direction L2 or the right direction R2 shown in FIG. 4. When the user performs the second joystick operation, the control unit 40 calculates a second image-processing control value in accordance with the second joystick operation. The second image-processing control value indicates a parameter of the image processing performed by the image-processing unit 57. For example, the control unit 40 calculates the second image-processing control value by using a similar method to that of calculating a bending control value.

Two types of tilting operations, in other words, a first tilting operation and a second tilting operation are prepared. The first tilting operation is associated with third image-processing control (second control). The third image-processing control is executed in order to control the image processing performed by the image-processing unit 57.

The user performs the first tilting operation by tilting the operation device 4 in the left direction L1 or the right direction R1 shown in FIG. 2. Alternatively, the user performs the first tilting operation by tilting the operation device 4 in the front direction F1 or the rear direction W1 shown in FIG. 3. When the user performs the first tilting operation, the control unit 40 calculates a third image-processing control value in accordance with the first tilting operation. The third image-processing control value indicates a parameter of the image processing performed by the image-processing unit 57. For example, the control unit 40 calculates the third image-processing control value by using a similar method to that of calculating a bending control value.

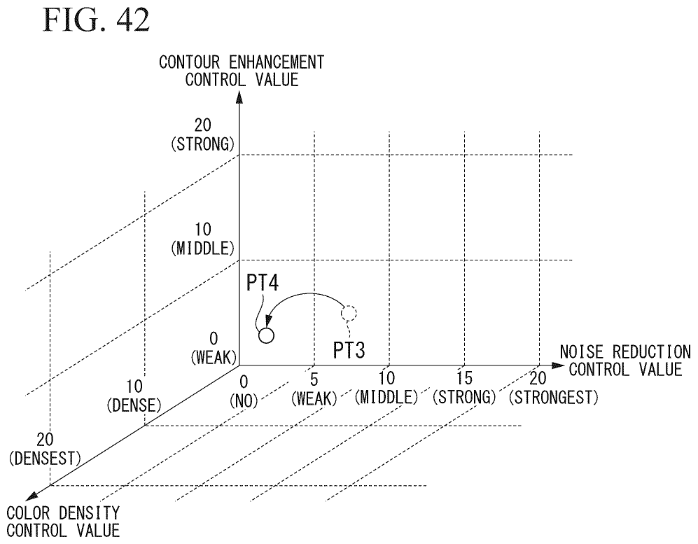

FIG. 42 is a graph showing an example of a first image-processing control value, a second image-processing control value, and a third image-processing control value. A first axis in the graph shown in FIG. 42 indicates the first image-processing control value, a second axis in the graph shown in FIG. 42 indicates the second image-processing control value, and a third axis in the graph shown in FIG. 42 indicates the third image-processing control value. In the example shown in FIG. 42, the first image-processing control value is a control value of the noise reduction, the second image-processing control value is a control value of the contour enhancement, and the third image-processing control value is a control value of the color density adjustment.

The second joystick operation, and the first tilting operation, the first image-processing control value, the second image-processing control value, and the third image-processing control value are values at a point PT3 before a user performs the first joystick operation. The user performs the first joystick operation and then performs the second joystick operation. Alternatively, the user performs the second joystick operation and then performs the first joystick operation. The user can perform the first tilting operation concurrently with the first joystick operation or the second joystick operation. The first image-processing control value, the second image-processing control value, and the third image-processing control value are values at a point PT4 after the user performs the first joystick operation, the second joystick operation, and the first tilting operation.

The second tilting operation is associated with (third control) control of switching types of image-processing control similarly to the first example. The user performs the second tilting operation by tilting the operation device 4 in the left direction L4 or the right direction R4 shown in FIG. 30.

When the control unit 40 determines to switch the types of image-processing control, the control unit 40 changes allocation of the image-processing control for the first control and the second control. For example, the control unit 40 allocates the third image-processing control to the first control associated with the first joystick operation, allocates the first image-processing control to the first control associated with the second joystick operation, and allocates the second image-processing control to the second control. When the user performs the first joystick operation, the control unit 40 calculates a third image-processing control value in accordance with the first joystick operation. When the user performs the second joystick operation, the control unit 40 calculates a first image-processing control value in accordance with the second joystick operation. When the user performs the first tilting operation, the control unit 40 calculates a second image-processing control value in accordance with the first tilting operation.

In the reference embodiment, a user can intuitively change a control value of image-processing by tilting the operation device 4. In addition, the user can change the control value of the image-processing and can finely adjust the control value by tilting the joystick 42.

### Seventh Embodiment

A seventh embodiment of the present invention will be described. In the seventh embodiment, the endoscope system 1 shown in FIG. 1 is used. In the seventh embodiment, the control unit 40 calculates a bending control value in accordance with movement of the housing 48 in a predetermined plane in a world coordinate system fixed in a three-dimensional space regardless of the posture of the housing 48.

In the first embodiment described above, the control unit 40 calculates a bending control value corresponding to the movement of the housing 48 in a predetermined plane in a coordinate system set in the housing 48. Hereinafter, a method of calculating a bending control value in the first embodiment will be described.

A first example of the method of calculating a bending control value will be described. In the first example, the insertion unit 2 is bent leftward or rightward in accordance with an operation of twisting the housing 48 by a user.

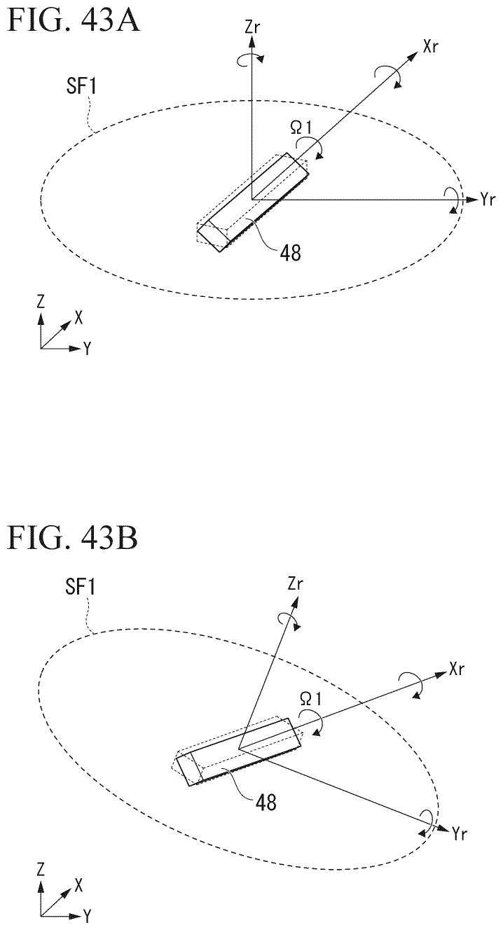

FIG. 43A shows an example of the posture of the housing 48. An X axis, a Y axis, a Z axis, an Xr axis, a Yr axis, and a Zr axis are shown in FIG. 43A. The X axis, the Y axis, and the Z axis are orthogonal to each other and constitute the world coordinate system fixed in the three-dimensional space. The Z axis extends in the opposite direction to the gravitational direction Dg. The Xr axis, the Yr axis, and the Zr axis are orthogonal to each other and constitute a local coordinate system fixed on the housing 48. The Xr axis corresponds to the axis AX1 shown in FIG. 2, the Yr axis corresponds to the axis AX2 shown in FIG. 2, and the Zr axis corresponds to the axis AX3 shown in FIG. 2.

The housing 48 includes a first surface on which the joystick 42 is disposed, a second surface opposite to the first surface, and four third surfaces connected to the first surface and the second surface. The third surfaces are side surfaces. The first surface and the second surface are broader than each of the third surfaces. The Xr axis and the Yr axis are parallel to the first surface and the second surface. The Zr axis is perpendicular to the first surface and the second surface.

A user operates the operation button 46 and inputs a reset instruction to the endoscope device 10. At this time, the Xr axis is parallel to the X axis, the Yr axis is parallel to the Y axis, and the Zr axis is parallel to the Z axis. In other words, a plane SF1 parallel to the first surface of the housing 48 is parallel to an XY plane including the X axis and the Y axis. Thereafter, the user twists the housing 48 by an angle Ω1 around the Xr axis. This movement of the housing 48 corresponds to the movement in a case in which the user tilts the housing 48 in the right direction R3 shown in FIG. 16A. The control unit 40 calculates a bending control value used for bending the insertion unit 2 rightward.

FIG. 43B shows another example of the posture of the housing 48. An X axis, a Y axis, a Z axis, an Xr axis, a Yr axis, and a Zr axis are shown in FIG. 43B similarly to FIG. 43A. A user operates the operation button 46 and inputs a reset instruction to the endoscope device 10. At this time, the Xr axis is not parallel to the X axis, the Yr axis is not parallel to the Y axis, and the Zr axis is not parallel to the Z axis. In other words, a plane SF1 parallel to the first surface of the housing 48 is not parallel to the XY plane. Thereafter, the user twists the housing 48 by the angle Ω1 around the Xr axis. The control unit 40 calculates a bending control value used for bending the insertion unit 2 rightward.

The bending control value in the example shown in FIG. 43B is the same as that in the example shown in FIG. 43A. A method of calculating a bending control value in the coarse movement mode is similar to that of calculating a bending control value shown in FIG. 16B. A bending control value in the coarse movement mode is ½*VA*(1+sin Ω1). A method of calculating a bending control value in the minute movement mode is similar to that of calculating a bending control value shown in FIG. 18B. A bending control value in the minute movement mode is ¼*VA*(2+sin Ω1).

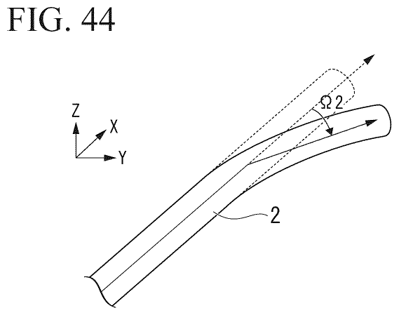

FIG. 44 shows an example in which the insertion unit 2 is bent. The insertion unit 2 is bent by an angle Ω2 in the direction of the Y-axis, that is, rightward, in accordance with the bending control value in the example shown in FIG. 43A or FIG. 43B.

A second example of the method of calculating a bending control value will be described. In the second example, the insertion unit 2 is bent leftward or rightward in accordance with an operation of moving the housing 48 leftward or rightward by a user.

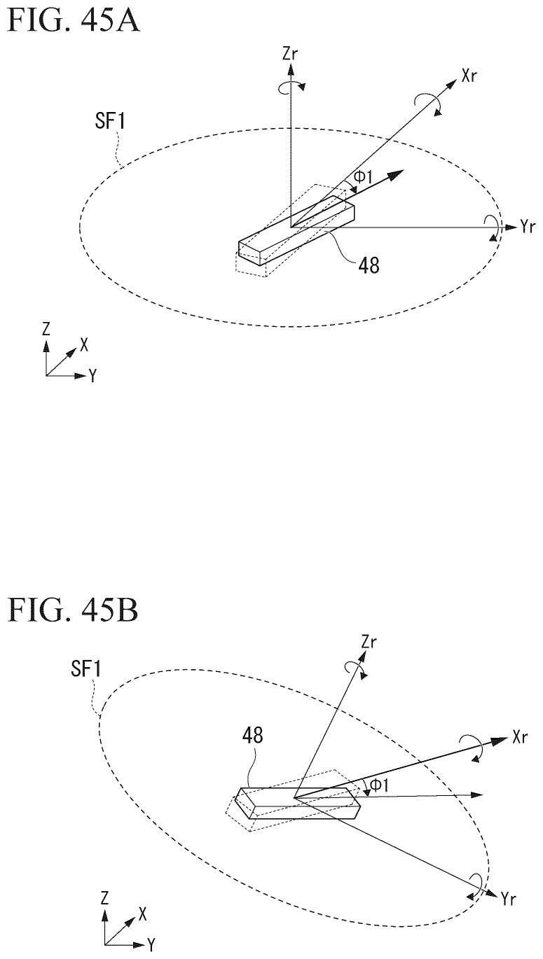

FIG. 45A shows an example of the posture of the housing 48. The X axis, the Y axis, the Z axis, the Xr axis, the Yr axis, and the Zr axis are shown in FIG. 45A similarly to FIG. 43A. A user operates the operation button 46 and inputs a reset instruction to the endoscope device 10. At this time, the Xr axis is parallel to the X axis, the Yr axis is parallel to the Y axis, and the Zr axis is parallel to the Z axis. In other words, a plane SF1 parallel to the first surface of the housing 48 is parallel to the XY plane. Thereafter, the user tilts the housing 48 by an angle Φ1 around the Zr axis in the direction of the Yr-axis. In other words, the user moves the housing 48 in the XY plane. The control unit 40 calculates a bending control value used for bending the insertion unit 2 rightward.

FIG. 45B shows another example of the posture of the housing 48. The X axis, the Y axis, the Z axis, the Xr axis, the Yr axis, and the Zr axis are shown in FIG. 45B similarly to FIG. 43A. A user operates the operation button 46 and inputs a reset instruction to the endoscope device 10. At this time, the Xr axis is not parallel to the X axis, the Yr axis is not parallel to the Y axis, and the Zr axis is not parallel to the Z axis. In other words, a plane SF1 parallel to the first surface of the housing 48 is not parallel to the XY plane. Thereafter, the user tilts the housing 48 by the angle Φ1 around the Zr axis in the direction of the Yr-axis. That is, the user moves the housing 48 in a plane that is not parallel to the XY plane. The control unit 40 calculates a bending control value used for bending the insertion unit 2 rightward.

The bending control value in the example shown in FIG. 45B is the same as that in the example shown in FIG. 45A. A method of calculating a bending control value in the coarse movement mode is the same as that of calculating a bending control value shown in FIG. 16B. A bending control value in the coarse movement mode is ½*VA*(1+sin Φ1). A method of calculating a bending control value in the minute movement mode is the same as that of calculating a bending control value shown in FIG. 18B. A bending control value in the minute movement mode is ¼*VA*(2+sin Φ1). The insertion unit 2 is bent in the direction of the Y-axis, that is, rightward, in accordance with the bending control value in the example shown in FIG. 45A or FIG. 45B similarly to the example shown in FIG. 44C.

A third example of the method of calculating a bending control value will be described. In the third example, the insertion unit 2 is bent upward or downward in accordance with an operation of moving the housing 48 upward or downward by a user.

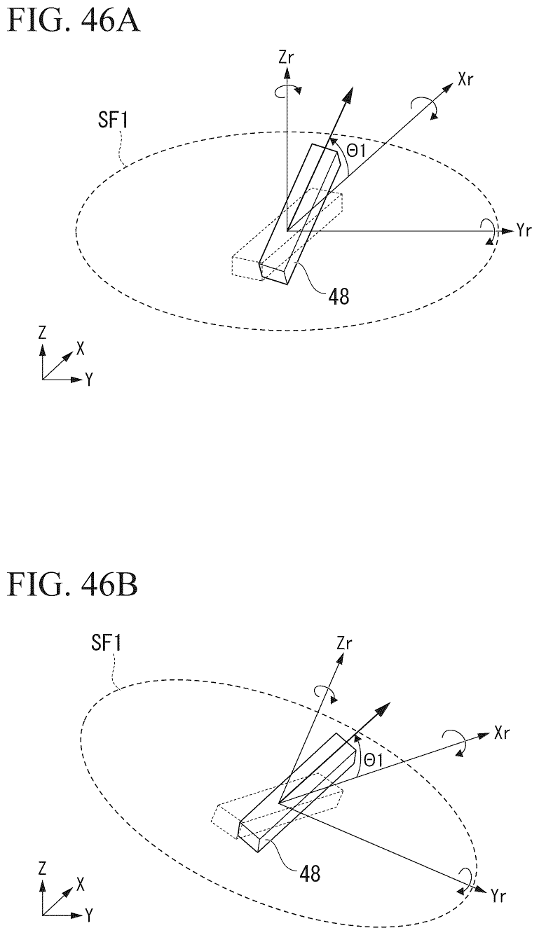

FIG. 46A shows an example of the posture of the housing 48. The X axis, the Y axis, the Z axis, the Xr axis, the Yr axis, and the Zr axis are shown in FIG. 46A similarly to FIG. 43A. A user operates the operation button 46 and inputs a reset instruction to the endoscope device 10. At this time, the Xr axis is parallel to the X axis, the Yr axis is parallel to the Y axis, and the Zr axis is parallel to the Z axis. In other words, a plane SF1 parallel to the first surface of the housing 48 is parallel to the XY plane. Thereafter, the user tilts the housing 48 by an angle θ1 around the Yr axis in the direction of the Zr-axis. In other words, the user moves the housing 48 in the XZ plane. The control unit 40 calculates a bending control value used for bending the insertion unit 2 upward.

FIG. 46B shows another example of the posture of the housing 48. The X axis, the Y axis, the Z axis, the Xr axis, the Yr axis, and the Zr axis are shown in FIG. 46B similarly to FIG. 43A. A user operates the operation button 46 and inputs a reset instruction to the endoscope device 10. At this time, the Xr axis is not parallel to the X axis, the Yr axis is not parallel to the Y axis, and the Zr axis is not parallel to the Z axis. In other words, a plane SF1 parallel to the first surface of the housing 48 is not parallel to the XY plane. Thereafter, the user tilts the housing 48 by the angle Θ1 around the Yr axis in the direction of the Zr-axis. In other words, the user moves the housing 48 in a plane that is not parallel to the XZ plane. The control unit 40 calculates a bending control value used for bending the insertion unit 2 upward.

The bending control value in the example shown in FIG. 46B is the same as that in the example shown in FIG. 46A. A method of calculating a bending control value in the coarse movement mode is the same as that of calculating a bending control value shown in FIG. 16B. A bending control value in the coarse movement mode is ½*VA*(1+sin Θ1). A method of calculating a bending control value in the minute movement mode is the same as that of calculating a bending control value shown in FIG. 18B. A bending control value in the minute movement mode is ¼*VA*(2+sin Θ1).

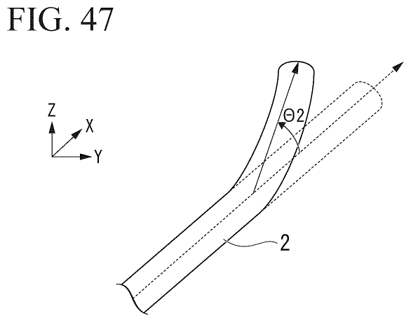

FIG. 47 shows an example in which the insertion unit 2 is bent. The insertion unit 2 is bent by an angle Φ2 in the direction of the Z-axis, that is, upward, in accordance with the bending control value in the example shown in FIG. 46A or FIG. 46B.

In the seventh embodiment, the control unit 40 calculates a bending control value corresponding to the movement of the housing 48 in a predetermined plane in the world coordinates system fixed in the three-dimensional space. Hereinafter, a method of calculating a bending control value in the seventh embodiment will be described.

A first example of the method of calculating a bending control value will be described. In the first example, the insertion unit 2 is bent leftward or rightward in accordance with an operation of moving the housing 48 leftward or rightward by a user.

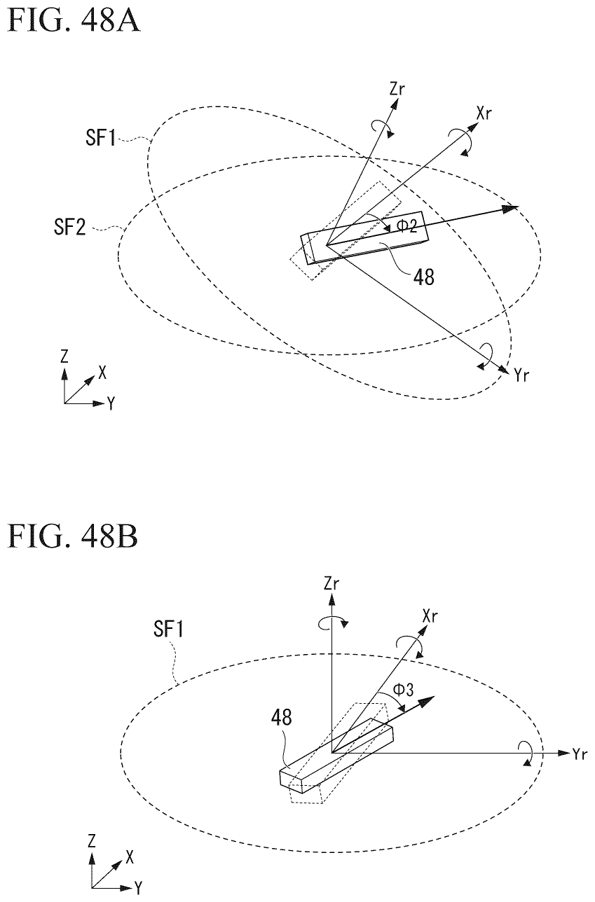

FIG. 48A shows an example of the posture of the housing 48. The X axis, the Y axis, the Z axis, the Xr axis, the Yr axis, and the Zr axis are shown in FIG. 48A similarly to FIG. 43A. A user operates the operation button 46 and inputs a reset instruction to the endoscope device 10. At this time, the Xr axis is not parallel to the X axis, the Yr axis is not parallel to the Y axis, and the Zr axis is not parallel to the Z axis. In other words, a plane SF1 parallel to the first surface of the housing 48 is not parallel to a XY plane SF2. Thereafter, the user tilts the housing 48 by an angle Φ2 around the Z axis in the direction of the Y-axis. In other words, the user moves the housing 48 in the XY plane SF2. The control unit 40 calculates a bending control value used for bending the insertion unit 2 rightward.

Since the Zr axis is not parallel to the Z axis, a straight line perpendicular to the first surface of the housing 48 is not parallel to a gravitational direction Dg. At this time, the control unit 40 corrects the digital value DI2 corresponding to the analog voltage AN2 output from the motion sensor 44.

FIG. 48B shows a posture of the housing 48 corresponding to the posture of the housing 48 shown in FIG. 48A. The X axis, the Y axis, the Z axis, the Xr axis, the Yr axis, and the Zr axis are shown in FIG. 48B similarly to FIG. 43A. The Xr axis is parallel to the X axis, the Yr axis is parallel to the Y axis, and the Zr axis is parallel to the Z axis. In other words, a plane SF1 parallel to the first surface of the housing 48 is parallel to the XY plane. In addition, before the user tilts the housing 48, a straight line perpendicular to the first surface of the housing 48 is parallel to a gravitational direction Dg.

The movement of the housing 48 shown in FIG. 48A corresponds to the movement when the user tilts the housing 48 shown in FIG. 48B by an angle Ø3 around the Zr axis in the direction of the Yr-axis. In the example shown in FIG. 48B, the straight line perpendicular to the first surface of the housing 48 is kept parallel to the gravitational direction Dg. The control unit 40 converts the digital value DI2 generated in accordance with the movement of the housing 48 shown in FIG. 48A into the digital value DI2 generated in accordance with the movement of the housing 48 shown in FIG. 48B. The control unit 40 corrects the digital value DI2 by executing this conversion.

The control unit 40 corrects the digital value DI2 in light of acceleration information in each of the Xr axis, the Yr axis, and the Zr axis in addition to posture information in each of the Xr axis, the Yr axis, and the Zr axis. Due to this, precision of the digital value DI2 increases. Specifically, the control unit 40 inputs information of 6 axes indicated by the digital value DI2 to a Madgwick filter and converts the information into stable posture information. Thereafter, the control unit 40 calculates coordinate values in the world coordinate system by using a general rotation matrix. The control unit 40 calculates a rotation amount in the XY plane by using the coordinate values. The control unit 40 may perform conversion to posture information by using another algorithm instead of the Madgwick filter.

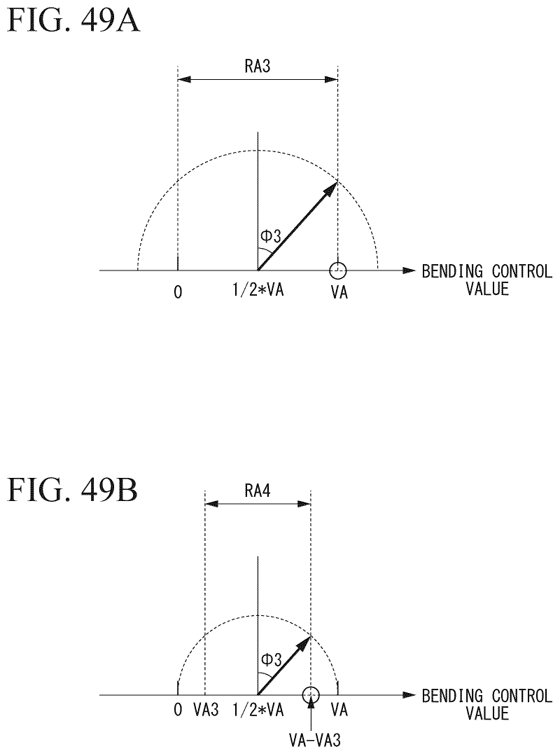

FIG. 49A shows a bending control value in the coarse movement mode. The control unit 40 calculates a bending control value in a range RA3 in accordance with the tilted state of the housing 48 shown in FIG. 48B. The minimum value of the range RA3 is 0, and the maximum value of the range RA3 is VA. When the bending control value is 0, the bending amount of the insertion unit 2 leftward in the coarse movement mode is the maximum. When the bending control value is VA, the bending amount of the insertion unit 2 rightward in the coarse movement mode is the maximum.

For example, the angle Φ3 shown in FIG. 48B is a maximum value set in advance and is less than 90 degrees. The bending control value (second control value) corresponding to the angle Φ3 is VA. At this time, the insertion unit 2 is bent in the direction of the Y-axis, that is, rightward, in accordance with the bending control value.

When a user tilts the housing 48 by an angle Φ2 that is the maximum angle in the opposite direction to the direction of the Yr-axis around the Zr axis, the bending control value (the second control value) is 0. This movement of the housing 48 corresponds to the movement when the user tilts the housing 48 shown in FIG. 48B by an angle Φ3 around the Zr axis in the opposite direction to the direction of the Yr-axis. At this time, the insertion unit 2 is bent leftward in accordance with the bending control value.

In the examples shown in FIG. 17A and FIG. 17B, the bending control value is the maximum when the user tilts the housing 48 by 90 degrees. On the other hand, in the examples shown in FIG. 48A and FIG. 49A, the bending control value is the maximum when the user tilts the housing 48 by the angle Φ2 that is less than 90 degrees. In the examples shown in FIG. 48A and FIG. 49A, the user can bend the insertion unit 2 with small movement of the housing 48.

FIG. 49B shows a bending control value in the minute movement mode. The control unit 40 calculates a bending control value in a range RA4 in accordance with the tilted state of the housing 48 shown in FIG. 48A. The minimum value of the range RA4 is VA3, and the maximum value of the range RA4 is (VA-VA3). When the bending control value is VA3, the bending amount of the insertion unit 2 leftward in the minute movement mode is the maximum. When the bending control value is (VA-VA3), the bending amount of the insertion unit 2 rightward in the minute movement mode is the maximum.

For example, the angle Φ3 shown in FIG. 48B is a maximum value set in advance and is less than 90 degrees. The bending control value (second control value) corresponding to the angle Φ3 is (VA-VA3). At this time, the insertion unit 2 is bent in the direction of the Y-axis, that is, rightward, in accordance with the bending control value.

When a user tilts the housing 48 by an angle Φ2 that is the maximum angle in the opposite direction to the direction of the Yr-axis around the Zr axis, the bending control value (second control value) is VA3. This movement of the housing 48 corresponds to the movement when the user tilts the housing 48 shown in FIG. 48B by an angle Φ3 in the opposite direction to the direction of the Yr-axis around the Zr axis. At this time, the insertion unit 2 is bent leftward in accordance with the bending control value.

In the examples shown in FIG. 17A and FIG. 19, the bending control value is the maximum when the user tilts the housing 48 by 90 degrees. On the other hand, in the examples shown in FIG. 48A and FIG. 49B, the bending control value is the maximum when the user tilts the housing 48 by the angle Φ2 that is less than 90 degrees. In the examples shown in FIG. 48A and FIG. 49B, the user can bend the insertion unit 2 with small movement of the housing 48.

When the plane SF1 parallel to the first surface of the housing 48 is not parallel to the XY plane SF2 as shown in FIG. 48A, the user moves the housing 48 in the XY plane SF2. The bending control value calculated in accordance with this movement of the housing 48 is the same as that calculated in accordance with the movement of the housing 48 in the XY plane SF2 after the user has held the operation device 4 such that the plane SF1 is parallel to the XY plane SF2. Therefore, the user can intuitively operate the operation device 4 regardless of the posture of the operation device 4.

A second example of the method of calculating a bending control value will be described. In the second example, the insertion unit 2 is bent upward or downward in accordance with an operation of moving the housing 48 upward or downward by a user.

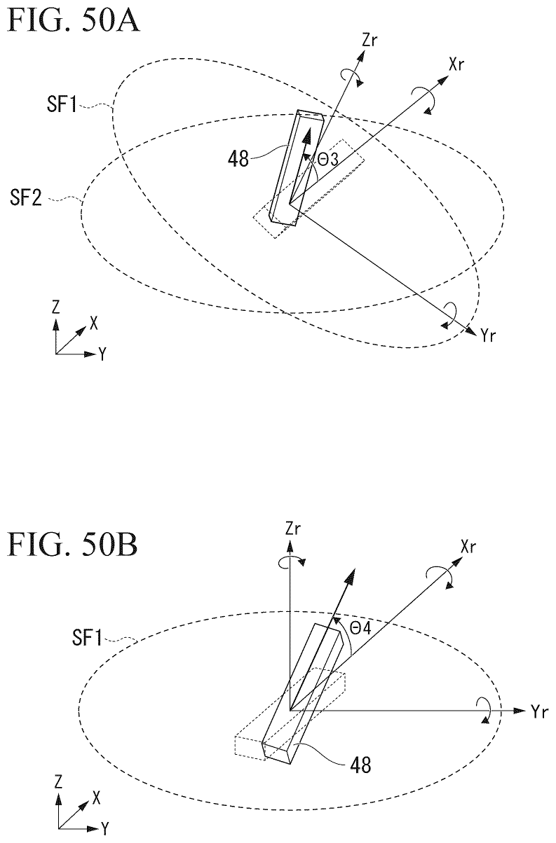

FIG. 50A shows an example of the posture of the housing 48. The X axis, the Y axis, the Z axis, the Xr axis, the Yr axis, and the Zr axis are shown in FIG. 50A similarly to FIG. 43A. A user operates the operation button 46 and inputs a reset instruction to the endoscope device 10. At this time, the Xr axis is not parallel to the X axis, the Yr axis is not parallel to the Y axis, and the Zr axis is not parallel to the Z axis. In other words, a plane SF1 parallel to the first surface of the housing 48 is not parallel to a XY plane SF2. Thereafter, the user tilts the housing 48 by an angle θ3 around the Y axis in the direction of the Z-axis. That is, the user moves the housing 48 in the XZ plane. The control unit 40 calculates a bending control value used for bending the insertion unit 2 upward.

Since the Zr axis is not parallel to the Z axis, a straight line perpendicular to the first surface of the housing 48 is not parallel to a gravitational direction Dg and a straight line parallel to the first surface is not perpendicular to the gravitational direction Dg. The control unit 40 corrects the digital value DI2 corresponding to the analog voltage AN2 output from the motion sensor 44 similarly to the example shown in FIG. 48A.

FIG. 50B shows a posture of the housing 48 corresponding to the posture of the housing 48 shown in FIG. 50A. The X axis, the Y axis, the Z axis, the Xr axis, the Yr axis, and the Zr axis are shown in FIG. 50B similarly to FIG. 43A. The Xr axis is parallel to the X axis, the Yr axis is parallel to the Y axis, and the Zr axis is parallel to the Z axis. In other words, a plane SF1 parallel to the first surface of the housing 48 is parallel to the XY plane. In addition, before the user tilts the housing 48, a straight line perpendicular to the first surface of the housing 48 is parallel to a gravitational direction Dg and a straight line parallel to the first surface is perpendicular to the gravitational direction Dg.

The movement of the housing 48 shown in FIG. 50A corresponds to the movement when the user tilts the housing 48 shown in FIG. 50B by an angle θ4 around the Y axis in the direction of the Z-axis. In the example shown in FIG. 50B, the straight line parallel to the first surface of the housing 48 is kept perpendicular to the gravitational direction Dg. The control unit 40 converts the digital value DI2 generated in accordance with the movement of the housing 48 shown in FIG. 50A into the digital value DI2 generated in accordance with the movement of the housing 48 shown in FIG. 50B. The control unit 40 corrects the digital value DI2 by executing this conversion.

Specifically, the control unit 40 inputs information of 6 axes indicated by the digital value DI2 to the Madgwick filter and converts the information into stable posture information. Thereafter, the control unit 40 calculates coordinate values in the world coordinate system bu using a general rotation matrix. The control unit 40 calculates a rotation amount in the XZ plane by using the coordinate values. The control unit 40 may perform conversion to posture information using another algorithm instead of the Madgwick filter.

A method of calculating a bending control value in the coarse movement mode is the same as that of calculating a bending control value shown in FIG. 49A. For example, the angle θ4 shown in FIG. 50B is a maximum value set in advance and is less than 90 degrees. The bending control value (second control value) corresponding to the angle θ4 is VA. At this time, the insertion unit 2 is bent in the direction of the Z-axis, that is, upward, in accordance with the bending control value.

When a user tilts the housing 48 by an angle θ3 that is the maximum angle in the opposite direction to the direction of the Z-axis around the Y axis, the bending control value (second control value) is 0. At this time, the insertion unit 2 is bent downward in accordance with the bending control value.

A method of calculating a bending control value in the minute movement mode is the same as that of calculating a bending control value shown in FIG. 49B. When the angle θ4 shown in FIG. 50B is the maximum value set in advance, the bending control value (second control value) corresponding to the angle θ4 is (VA-VA3). At this time, the insertion unit 2 is bent in the direction of the Z-axis, that is, upward, in accordance with the bending control value.

When a user tilts the housing 48 by an angle θ3 that is the maximum angle in the opposite direction to the direction of the Z-axis around the Y axis, the bending control value (second control value) is VA3. At this time, the insertion unit 2 is bent downward in accordance with the bending control value.

In the examples shown in FIG. 50A and FIG. 50B, similarly to the examples shown in FIG. 48A and FIG. 48B, a user can bend the insertion unit 2 with small movement of the housing 48.

When the plane SF1 parallel to the first surface of the housing 48 is not parallel to the XY plane SF2 as shown in FIG. 50A, the user moves the housing 48 in the XZ plane. The bending control value calculated in accordance with this movement of the housing 48 is the same as that calculated in accordance with the movement of the housing 48 in the XZ plane after the user has held the operation device 4 such that the plane SF1 is parallel to the XY plane SF2. Therefore, the user can intuitively operate the operation device 4 regardless of the posture of the operation device 4.

Each aspect of the present invention may include the following modified example. The joystick 42 (user interface) is disposed on the surface of the housing 48. The control unit 40 converts the digital value DI2 corresponding to the analog voltage AN2 (second signal) generated in accordance with the movement of the operation device 4 in a first state into the digital value DI2 corresponding to the analog voltage AN2 (second signal) generated in accordance with the movement of the operation device 4 in a second state. A straight line (axis AX3) perpendicular to the surface of the housing 48 is not parallel to the gravitational direction Dg in the first state. The straight line is parallel to the gravitational direction Dg in the second state.

Each aspect of the present invention may include the following modified example. A straight line (axis AX2) perpendicular to the surface of the housing 48 is not perpendicular to the gravitational direction Dg in the first state. The straight line is perpendicular to the gravitational direction Dg in the second state.

In the seventh embodiment, the control unit 40 calculates a bending control value corresponding to the movement in the housing 48 in the XY plane or the YZ plane regardless of the posture of the housing 48. The endoscope system 1 can support a user's intuitive operation of the operation device 4.

While preferred embodiments of the invention have been described and shown above, it should be understood that these are examples of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. An endoscope system, comprising:
an endoscope device including a bendable insertion unit;
an operation device including:
a housing;
a user interface that is disposed on the housing and generates a first signal in accordance with a state of the user interface;
wherein the state changes when the user interface touches an object; and
a motion sensor configured to generate a second signal in accordance with physical movement of the operation device; and
a processor configured to:
calculate a first control value used in first control of the endoscope device based on the first signal; and
calculate a second control value used in second control of the endoscope device based on the second signal,
wherein at least one of the first control and the second control is executed to bend the insertion unit.

2. The endoscope system according to claim 1,
wherein the first control and the second control are executed to bend the insertion unit.

3. The endoscope system according to claim 2,
wherein a maximum value of a bending amount of the insertion unit in the second control is different from a maximum value of the bending amount of the insertion unit in the first control.

4. The endoscope system according to claim 2,
wherein the processor is configured to:
calculate the first control value in a first period during which the first signal is generated without the second signal being generated; and
calculate the first control value without calculating the second control value in a second period during which the first signal and the second signal are generated after the first period.

5. The endoscope system according to claim 2,
wherein the processor is configured to:
calculate the second control value in a first period during which the second signal is generated without the first signal being generated; and
calculate the second control value without calculating the first control value in a second period during which the first signal and the second signal are generated after the first period.

6. The endoscope system according to claim 2,
wherein a range of a bending amount of the insertion unit in the second control is different from a range of the bending amount of the insertion unit in the first control.

7. The endoscope system according to claim 1,
wherein the movement contains rotation of the operation device.

8. The endoscope system according to claim 7,
wherein the motion sensor is configured to generate a third signal in accordance with the rotation of the operation device, and
wherein the processor is configured to:
allocate one of control of a first function and control of a second function to the first control based on the third signal; and
allocate the other of the control of the first function and the control of the second function to the second control based on the third signal.

9. The endoscope system according to claim 7,
wherein the operation device has an elongated shape,
wherein the motion sensor is configured to generate the second signal in accordance with the rotation of the operation device around an axis perpendicular to a longitudinal direction of the operation device, and
wherein the second control is executed to bend the insertion unit.

10. The endoscope system according to claim 7,
wherein the operation device includes a sensor that outputs a signal in accordance with whether a user is holding the operation device with a left hand or is holding the operation device with a right hand,
wherein the processor is configured to:
determine whether the user is holding the operation device with the left hand or is holding the operation device with the right hand based on the signal output from the sensor;
calculate the second control value by using first information indicating a relationship between an amount of the rotation and the second control value when it is determined that the user is holding the operation device with the left hand; and
calculate the second control value by using second information indicating a relationship between the amount of the rotation and the second control value when it is determined that the user is holding the operation device with the right hand, and
wherein the second information is different from the first information.

11. The endoscope system according to claim 1, further comprising an insertion device configured to insert a distal end of the insertion unit into a subject and pulls the distal end out of the subject,
wherein one of the first control and the second control is executed to bend the insertion unit, and
wherein the other of the first control and the second control is executed to control insertion and pullout of the distal end.

12. The endoscope system according to claim 11,
wherein the operation device has an elongated shape,
wherein the motion sensor is configured to generate a third signal in accordance with rotation of the operation device around an axis parallel to a longitudinal direction of the operation device,
wherein the insertion device rotates the insertion unit around a center axis of the insertion unit, and
wherein the processor is configured to calculate, based on the third signal, a third control value used to control rotation of the insertion unit.

13. The endoscope system according to claim 1,
wherein the operation device includes a switch disposed on the housing,
wherein a state of the switch is switched between a first state and a second state,
wherein the second signal is valid when the state of the switch is the first state,
wherein the second signal is invalid when the state of the switch is the second state, and
wherein the processor is configured to calculate the second control value only when the second signal is valid.

14. The endoscope system according to claim 1,
wherein the user interface includes a movable member disposed on the housing.

15. The endoscope system according to claim 1,
wherein the operation device includes the processor.

16. The endoscope system according to claim 1,
wherein the endoscope device includes the processor.

17. The endoscope system according to claim 1,
wherein the endoscope device includes an image sensor configured to generate an image based on an optical image acquired by the insertion unit, and
wherein one of the first control and the second control is executed to control image processing of changing a state of the image.

18. The endoscope system according to claim 1,
wherein the processor is configured to select one of three or more control values corresponding to three or more bending amounts of the insertion unit as the first control value based on the first signal.

19. The endoscope system according to claim 1,
wherein the user interface is disposed on a surface of the housing,
wherein the processor is configured to convert the second signal generated in accordance with the movement of the operation device in a first state into the second signal generated in accordance with the movement of the operation device in a second state,
wherein a straight line perpendicular to the surface is not parallel to a gravitational direction in the first state, and
wherein the straight line is parallel to the gravitational direction in the second state.

20. The endoscope system according to claim 1, wherein the user interface is disposed on a surface of the housing,
wherein the processor is configured to convert the second signal generated in accordance with the movement of the operation device in a first state into the second signal generated in accordance with the movement of the operation device in a second state,
wherein a straight line parallel to the surface is not perpendicular to a gravitational direction in the first state, and
wherein the straight line is perpendicular to the gravitational direction in the second state.

21. An operation device, comprising:
a housing;
a user interface that is disposed on the housing and generates a first signal in accordance with a state of the user interface,
wherein the state changes when the user interface touches an object; and
a motion sensor configured to generate a second signal in accordance with physical movement of the operation device,
wherein the first signal is used to calculate a first control value used in first control of an endoscope device including a bendable insertion unit,
wherein the second signal is used to calculate a second control value used in second control of the endoscope device, and
wherein at least one of the first control and the second control is executed to bend the insertion unit.
